(19) United States
(12) Patent Application Publication
(10) Pub. No.: US 2025/0066306 A1
(43) Pub. Date: Feb. 27, 2025

(54) COMPOUNDS FOR TREATING MICROBIAL INFECTIONS, METHODS OF MAKING AND USES THEREOF

(71) Applicants: McMaster University, Hamilton (CA); University of Florida Research Foundation, Gainsville, FL (US); UNITED STATES GOVT - THE DEPT OF VETERANS AFFAIRS, Washington, DC (US); University of Pittsburgh, Pittsburgh, PA (US)

(72) Inventors: James McNulty, St. Catherines (CA); Chanti Babu Dokuburra, Hamilton (CA); Leonardo D'Aiuto, Pittsburgh, PA (US); Vishwajit Nimgaonkar, Pittsburgh, PA (US); David C Bloom, Gainsville, FL (US); Maribeth Wesesky, Pittsburgh, PA (US); Joel Wood, Pittsburgh, PA (US)

(21) Appl. No.: 18/738,421

(22) Filed: Jun. 10, 2024

Related U.S. Application Data
(60) Provisional application No. 63/472,002, filed on Jun. 9, 2023.

(51) Int. Cl. C07D 221/12; A61K 31/473; A61K 31/522; A61P 31/14; A61P 31/22

(52) U.S. Cl. CPC C07D 221/12; A61K 31/473; A61K 31/522; A61P 31/14; A61P 31/22

(57) ABSTRACT

This application relates to compounds of Formula (I) that activates the integrated stress response, compositions comprising these compounds and methods of use thereof, for example, for treating diseases, disorders or conditions treatable by activating the integrated stress response.

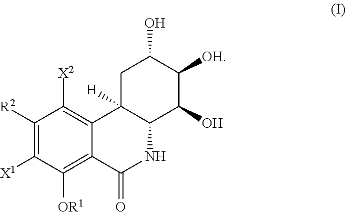

### CROSS-REFERENCE TO RELATED APPLICATIONS

The present application claims the benefit of priority from U.S. provisional patent application No. 63/472,002 filed on Jun. 9, 2023, the contents of which are incorporated herein by reference in their entirety.

### REFERENCE TO SQUENCE LISTING

A computer readable form of the Sequence Listing “8500-011US_SequenceListing.xml” (4,619 bytes), submitted via Patent Center and created on Sep. 16, 2024, is herein incorporated by reference.

### FIELD

The present disclosure relates to compounds that activate the integrated stress response, compositions comprising these compounds and methods of use thereof for example, for treating diseases, disorders or conditions treatable by activating the integrated stress response, alone or in combination with other pharmaceuticals.

### BACKGROUND

The use of natural products in the discovery of initial hit compounds is a well-established strategy in drug discovery.¹ This tradition that has gained some traction towards the development of structurally novel anti-coronavirus leads,^{1c} approved and current investigational therapeutics are repurposed and/or designed agents that target viral proteins such as protease or RNA polymerase inhibition.^{1d} Alkaloids isolated from various members of the Amaryllidaceae plant family have proven of much potential in view of the demonstrated potent anticancer,² antiviral³ and acetylcholinesterase activity. The antiviral activity of narciclasine (1) (shown in FIG. 1) and a wide range of alkaloids of this lycorane sub-class was reported three decades ago.^{3a} The potent antiviral activity of 1-deoxypancratistatin (2a ) and trans-dihydrolycoricidine (2b) (shown in FIG. 1) to DNA viruses such as Herpes (HSV-1, VZV) and RNA viruses including Zika (ZIKV) has been recently reported.^{3c-d,4b} The related alkaloid lycorine (3) (shown in FIG. 1) has been shown to significantly inhibit replication of the SARS-associated coronavirus.^{3e} The overall RNA-virus activity of these alkaloids and potential development of novel anti-coronavirus drugs has recently been reviewed.^{3f}

Despite the availability of compounds that possess activity to both DNA and RNA viruses^{3c-d,4b}, there is a need for compounds with potent and selective activity without host cell cytotoxicity (or with very low level of cell cytotoxicity). Furthermore, there is a need for antimicrobial agents that are not prone to selection toward drug resistance.

The background herein is included solely to explain the context of the disclosure. This is not to be taken as an admission that any of the material referred to was published, known, or part of the common general knowledge as of the priority date.

### SUMMARY

The present disclosure describes the asymmetric synthesis of the ring-A mono-substituted C9-OMe (5), C9-OH (6), C7-OMe (7), and C7-OH (Ia) analogs of Amryllidaceae alkaloids, containing the fully functionalized ring-C (FIG. 2). Antiviral activity studies showed that the 7-hydroxyl analog Ia, demonstrates antiviral activity to HSV-1 as well as potent and selective anti-coronaviral activity to the SARSCoV2 coronavirus. Given the activity observed to both DNA and RNA viruses, the target and mechanism of action of compound Ia are also reported herein. Compound Ia activates the eukaryotic integrated stress response (ISR) and resulting signaling network, inducing autophagy, and upregulates the sirtuin signaling pathway and TP53, with activation of innate immunity. Upregulation of these synergistic antiviral defenses in the host cell explains the broad-spectrum antiviral activity observed. These processes are not dependent on interactions with highly mutable viral structural proteins and non-structural targets such as proteases, thus compound Ia represents an important lead towards advancing a stable, broad-spectrum antiviral agent.

In accordance with an aspect, there is provided a compound of Formula (I) or a pharmaceutically acceptable salt, solvate and/or prodrug thereof:

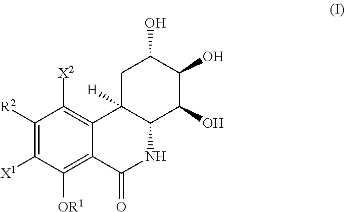

wherein
R¹ is selected from H, Me and Bn;
R² is selected from H, OH, OMe, OBn, CF₃ and OCF₃;
X¹ is selected from H, F, Cl, Br and I; and
X² is selected from H, F, Cl, Br and I.

In an embodiment, the compound is selected from

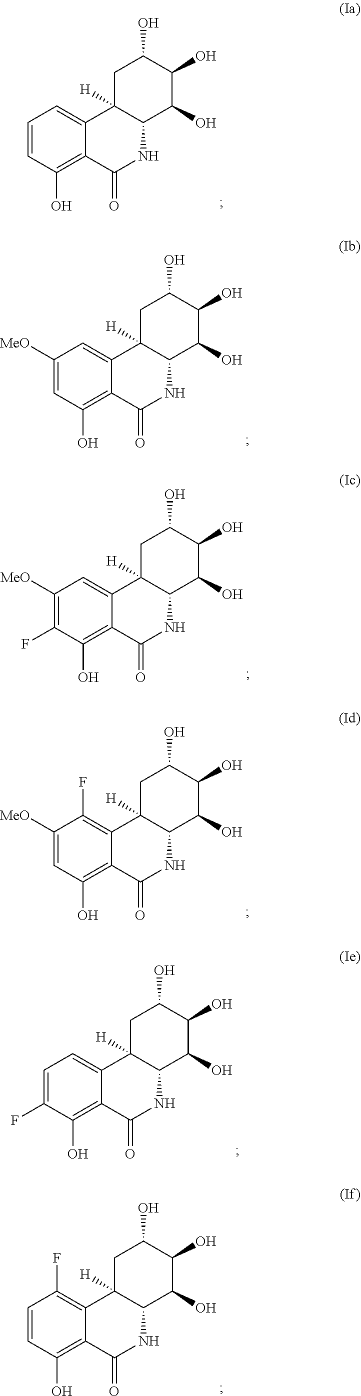 [SMILES: O=C1N[C@H]2[C@H](O)[C@H](O)[C@@H](O)C[C@@H]2c2cccc(O)c21 ; COc1cc(O)c2c(c1)[C@H]1C[C@H](O)[C@@H](O)[C@@H](O)[C@@H]1NC2=O ; COc1cc2c(c(O)c1F)C(=O)N[C@H]1[C@H](O)[C@H](O)[C@@H](O)C[C@H]21 ; COc1cc(O)c2c(c1F)[C@H]1C[C@H](O)[C@@H](O)[C@@H](O)[C@@H]1NC2=O ; O=C1N[C@H]2[C@H](O)[C@H](O)[C@@H](O)C[C@@H]2c2ccc(F)c(O)c21 ; O=C1N[C@H]2[C@H](O)[C@H](O)[C@@H](O)C[C@@H]2c2c(F)ccc(O)c21]

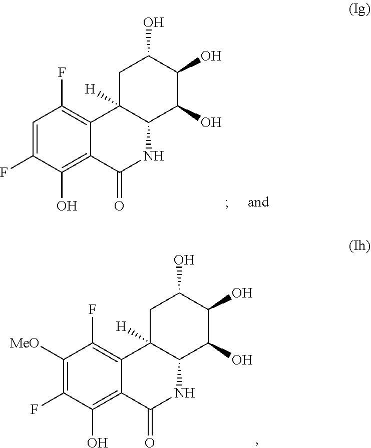 [SMILES: O=C1N[C@H]2[C@H](O)[C@H](O)[C@@H](O)C[C@@H]2c2c(F)cc(F)c(O)c21 ; COc1c(F)c(O)c2c(c1F)[C@H]1C[C@H](O)[C@@H](O)[C@@H](O)[C@@H]1NC2=O]

or a pharmaceutically acceptable salt, solvate and/or prodrug thereof.

In an embodiment, there is provided a pharmaceutical composition comprising one or more compounds of Formula (I), or a pharmaceutically acceptable salt, solvate and/or prodrug thereof, and one or more pharmaceutically acceptable carriers.

In an embodiment, the present disclosure includes a method of treating a disease, disorder or condition treatable by activating the integrated stress response via upregulation of eukaryotic initiation factor 2α (eIF2α) is provided, the method comprising administering a therapeutically effective amount of one or more compounds of Formula (I) or a pharmaceutically acceptable salt, solvate and/or prodrug thereof, to a subject in need thereof.

In an embodiment, the disease, disorder or condition is a viral infection.

In an embodiment, the viral infection comprises infection with DNA or RNA viruses.

In an embodiment, the DNA virus is HSV-1.

In an embodiment, the RNA virus is SARSCoV-2.

In an embodiment, the one or more compounds of Formula (I) are administered by ocular, topical, transdermal, or oral administration, or by inhalation or injection.

In an embodiment, the one or more compounds are administered in combination with one or more therapeutic agents.

In an embodiment, the one or more therapeutic agents is an antiviral agent. In an embodiment, the antiviral agent is acyclovir.

In an embodiment, a method of activating the integrated stress response via upregulation of eukaryotic initiation factor 2α (eIF2α) in a cell, either in a biological sample or in a subject is provided, comprising administering a therapeutically effective amount of one or more compounds of Formula (I), or a pharmaceutically acceptable salt, solvate and/or prodrug thereof, to a cell in need thereof.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### DRAWINGS

Certain embodiments of the disclosure will now be described in greater detail with reference to the attached drawings in which:

FIG. 1 shows A: Structure of known natural amaryllidaceae constituents with potent antiviral activity, narciclasine (1), 1-deoxypancratistatin (trans-dihydronarciclasine) (2a ), trans-dihydrolycoricidine (2b), lycorine (3) and the inactive synthetic 10b-aza analog (4). B: The exemplary trans-dihydronarciclasine analogs 5, 6, 7 and Ia of the present application and proposed retrosynthesis via the cyclohexane derivative 9, which could be derived from cinnamaldehyde derivative 10 and azido acetone 11 in exemplary embodiments of the disclosure.

FIG. 2 shows A: Synthesis of exemplary triacetates of trans-dihydronarciclasine: Pd/C loading and time a) 1. (CH₃CH₂O)₂CHCH₂P(CH₂CH₂CH₃)₃Br, NaH, THF; 2. 1M HCl, RT, 95%; b) 11, CH₂Cl₂, (R)-diphenylprolinol TMS ether, quinidine, −10° C.-RT, 55%; C) H₂, 15 Mol % Pd/C MeOH, 32 h, RT, 88%; d) 1. DIPEA, MsCl, CH₂Cl₂, 10 h, RT, 2. Li(tBuO)₃AlH, THF, 20 h, 0° C.-RT, 95%; e) 1. mCPBA, NaHCO₃, CH₂Cl₂, 24 h, RT; 2. NaBz/H₂O, 16 h, 90-95° C.; 3. Ac₂O, Py, 16 h, RT, over 3 steps 77%; f) Tf₂O, DMAP, CH₂Cl₂, 16 h, 0° C.-RT. B: Formation of non-symmetrical dimer 18: C′) H₂, 10 Mol % Pd/C, DMDC, MeOH, 16 h, RT in exemplary embodiments of the disclosure.

FIG. 3 shows synthesis of exemplary trans-dihydronarciclasine derivatives (5, 6, 7 and Ia) in exemplary embodiments of the disclosure. a) K₂CO₃, MeOH, 95%; b) AlCl₃/TBAI, CH₂Cl₂:C₆H₆ (1:1) 88%; C) K₂CO₃, MeOH, 94%; d) K₂CO₃, MeOH, 96%; e) AlCl₃/TBAI, CH₂Cl₂:C₆H₆ (1:1) 89%; f) K₂CO₃, MeOH, 96%.

FIG. 4 shows compounds 2b and Ia (compound 8) inhibit to a different extent HSV-1 in human iPSC-derived NPCs in an exemplary embodiment of the disclosure. (A) Flow cytometry (FC) analysis of uninfected and infected NPC lines 01SD and 9001 in the presence or absence of ACV, 2b and its derivatives 6, 7 and Ia (compound 8) using an engineered HSV-1 construct DualF, expressing EGFP from an IE gene promoter and RFP from a late gene promoter. NPCs were infected at a multiplicity of infection of 0.1. The aforementioned compounds were added at two hours p.i., and cultures were analyzed at day 2 p.i.. The fractions of cells showing fluorescence of the appropriate color are indicated. (B) Cytotoxycity of 2b (right columns), Ia (middle columns, compound 8) and ACV (left columns) at varying concentrations in uninfected 01SD NPC cultures was assessed by FC using fixable viability dye. The data represent an average of six independent experiments. Error bars represent standard deviations.

FIG. 5 shows characterization of 9001 brain organoids (A) Hematoxylin and eosin staining of 9-weeks old organoid in an exemplary embodiment of the application. (B-E) Immunostaining of 8 microns thick sections of organoids with antibodies recognizing CALBINDIN (B), CUX2/TUJI (C), SOX2 (D), CTIP2 (E). Nuclei were counterstained with Hoechst 33342. Scale bar is 50 μm in (A), 75 μm in (B), and 250 μm in (C-E).

FIG. 6 shows the anti-HSV-1 efficacy of the exemplary compound Ia comparable to 2b and ACV, in human brain organoids in exemplary embodiments of the disclosure. Fourteen-week old brain organoids generated from 9001 and 01SD iPSC-lines were infected individually with HSV-1 DualF construct (6000 pfu/organoid). Two hours after the infection the inocula were removed, the organoids washed and cultured in the presence of the tested compounds. The expression of the fluorescent reported genes EGFP and RFP was visually inspected under a fluorescent microscope at day 3 p.i. N=3 for each condition. Scale bar: 50 μm.

FIG. 7 shows antiviral efficacy and cytotoxicity of exemplary compound Ia against HSV-1 in human brain organoids in exemplary embodiments of the disclosure. (A) Sixteen-week old 9001 and 01SD brain organoids were infected individually with HSV-1 DualF construct (6000 pfu/organoid). After 2 hours the inocula were removed and Ia was added to varying concentrations (100 nM-50 M). The IC50 was estimated by determining the corrected total cell fluorescence (CTCF) obtained by measuring EGFP fluorescence in organoids using ImageJ and normalizing for background using equation CTCF=integrated density−(area of the organoid x mean fluorescence of background readings). (b) Cytotoxicity of compound Ia without HSV-1 infection was assessed using calcein-AM. The fluorescence of calcein-AM in each organoid was measured and CTCF was determined as described above. The data represent an average of six independent experiments. Error bars represent standard deviations. No significant difference were found between conditions. Scale bar: 50 μM.

FIG. 8 shows comparison of quantitative reverse transcription PCR (RT-qPCR) and RNA sequencing assays for selected human transcripts in an exemplary embodiment of the disclosure. RT-qPCR was used to estimate expression of four genes that showed significant Ia (compound 8) and 2b-induced changes in transcript levels in the RNA sequencing experiments. Values shown as means and error bars denote standard deviations.

FIG. 9 shows human gene pathways significantly altered by compounds 2b and Ia (compound 8) in exemplary embodiments of the disclosure. Differentially Expressed Genes (DEG) measured by comparing 2b- and Ia (compound 8)-treated NPCs vs. vehicle-treated NPCs were subjected to Ingenuity Pathway Analysis (IPA). The top 10 pathways showing the greatest statistical significance (Fisher's Exact Test p-value) are shown. Activation (+ve z-score, orange bars) or inhibition (−ve z-score, blue bars) of each pathway is a measure of experimentally determined gene expression changes reported in the literature. Data were analyzed through the use of QIAGEN Ingenuity® Pathway Analysis. The intensity of color indicates the degree of activation/inhibition. Pathways without an activity pattern are in grey. Ratios (orange square above each pathway) represent the degree of overlap between DEG and all members of a given pathway. Y-axis on the right indicates ratio values.

FIG. 10 shows activation of the integrated stress response in compounds 2b- and Ia-treated NPCs in exemplary embodiments of the disclosure. (A) NPCs derived from 9001 iPSC lines were exposed to compounds 7, Ia (compound 8), and 2b (10 μM). After 24 hours, cells were fixed and stained with an antibody that detects eIF2α phosphorylated on Serine 51 (EIF2S1). Nuclei were counterstained with Hoechst 33342, and phosphorylation was assessed by fluorescence microscopy. (B) The corrected total cell fluorescence (CTCF) was obtained by measuring EIF2S1 fluorescence in cells where a fluorescent signal could be detected and normalizing for cellular area using equation CTCF=integrated density−(cellular area×mean fluorescence of background readings) (C-D). Up-regulation of the EIF2A kinase EIF2AK3 (C) and the activated transcription factor 4 (ATF4) (D) in 2b- and Ia (compound 8)-treated NPCs. (E) Proposed mechanism of action of 2b and Ia (compound 8).

FIG. 11 shows in vivo antiviral activity in an exemplary embodiment of the disclosure. ND4 swiss mice were infected by corneal scarification with 1×10⁵ pfu of HSV-1 strain 17syn+. Eyes were swabbed daily for 4 days and assayed for infectious virus by standard plaque assay. Exemplary compound Ia (code R799) dramatically reduces HSV-1 shedding in the mouse eye.

FIG. 12 shows 1H NMR spectra of exemplary compounds and intermediates used in their synthesis in an exemplary embodiment of the disclosure.

FIG. 13 shows chiral HPLC analyses of intermediates used in the preparation of exemplary compounds of the disclosure.

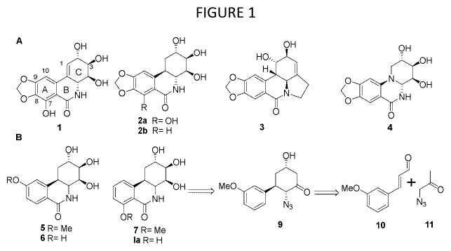

### DETAILED DESCRIPTION

### I. Definitions

Unless otherwise indicated, the definitions and embodiments described in this and other sections are intended to be applicable to all embodiments and aspects of the present disclosure herein described for which they are suitable as would be understood by a person skilled in the art. It is also to be understood that the terminology used herein is for the purpose of describing particular aspects only and is not intended to be limiting.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.”

The term “compound(s) of the application” as used herein means a compound of Formula I, including pharmaceutically acceptable salts, solvates and/or prodrugs thereof.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with the treatment of subjects.

The term “solvate” as used herein means a compound, or a salt and/or prodrug of a compound, wherein molecules of a suitable solvent are incorporated in the crystal lattice. A suitable solvent is physiologically tolerable at the dosage administered.

The term “prodrug” as used herein means a compound, or salt and/or solvate of a compound, that, after administration, is converted to an active drug.

The term “treating” or “treatment” as used herein means an approach for obtaining beneficial or desired results, including clinical results. Beneficial or desired clinical results can include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” or “treatment” can also mean prolonging survival. Treatment methods comprise administering to a subject a therapeutically effective amount of one or more of the compounds of the application and optionally consist of a single administration, or alternatively comprise a series of administrations.

As used herein, the term “effective amount” or “therapeutically effective amount” means an amount of a compound, or one or more compounds, of the application that is effective, at dosages and for periods of time necessary to achieve the desired result.

It will be understood that any component defined herein as being included may be explicitly excluded by way of proviso or negative limitation, such as any specific compounds or method steps, whether implicitly or explicitly defined herein.

### II. Compounds and Compositions of the Application

The present application includes a compound of Formula I, or a pharmaceutically acceptable salt, solvate and/or prodrug thereof:

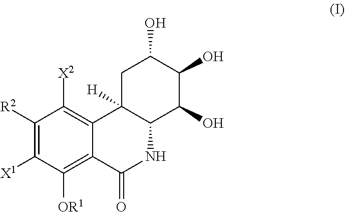

wherein
R¹ is selected from H, Me and Bn;
R² is selected from H, OH, OMe, OBn, CF₃ and OCF₃;
X¹ is selected from H, F, Cl, Br and I; and
X² is selected from H, F, Cl, Br and I.

In some embodiments, R¹ is H.

In some embodiments, R² is selected from H and OCH₃. In some embodiments, R² is H.

In some embodiments, X¹ is selected from H and F. In some embodiments, X¹ is H.

In some embodiments, X² is selected from H and F. In some embodiments, X² is H.

In some embodiments, R¹, R², X¹ and X² are H.

In some embodiments, the compounds of Formula I are selected from the compounds listed below, or a pharmaceutically acceptable salt, solvate and/or prodrug thereof:

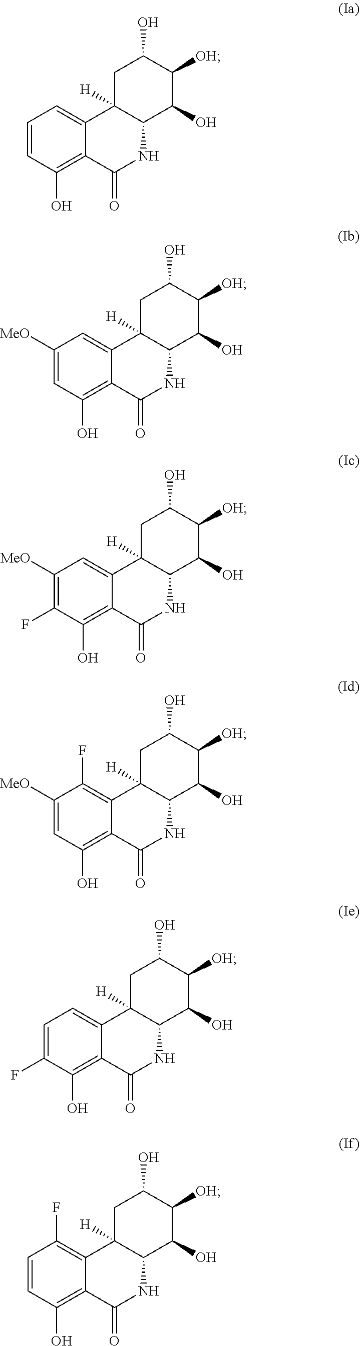

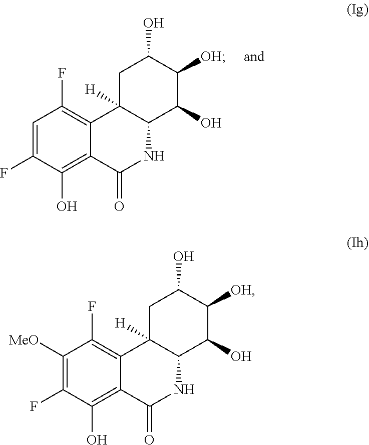 [SMILES: O=C1N[C@H]2[C@H](O)[C@H](O)[C@@H](O)C[C@@H]2c2c(F)cc(F)c(O)c21 ; "and" ; COc1c(F)c(O)c2c(c1F)[C@H]1C[C@H](O)[C@@H](O)[C@@H](O)[C@@H]1NC2=O]

In some embodiments, the pharmaceutically acceptable salt is an acid addition salt or a base addition salt. The selection of a suitable salt may be made by a person skilled in the art. Suitable salts include acid addition salts that may, for example, be formed by mixing a solution of a compound with a solution of a pharmaceutically acceptable acid such as hydrochloric acid, sulfuric acid, acetic acid, trifluoroacetic acid, or benzoic acid. Additionally, acids that are generally considered suitable for the formation of pharmaceutically useful salts from basic pharmaceutical compounds are discussed, for example, by P. Stahl et al, Camille G. (eds.) and Handbook of Pharmaceutical Salts. Properties, Selection and Use. (2002) Zurich: Wiley VCH; S. Berge et al, Journal of Pharmaceutical Sciences 1977 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In some embodiments, exemplary acid addition salts also include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and the like. In some embodiments, the mono- or di-acid salts are formed and such salts exist in either a hydrated, solvated or substantially anhydrous form. In general, acid addition salts are more soluble in water and various hydrophilic organic solvents and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Exemplary organic bases are isopropylamine, diethylamine, ethanolamine, trimethylamine, dicyclohexylamine, choline and caffeine. The selection of the appropriate salt may be useful, for example, so that an ester functionality, if any, elsewhere in a compound is not hydrolyzed. The selection criteria for the appropriate salt will be known to one skilled in the art. In some embodiments, exemplary basic salts also include ammonium salts, alkali metal salts such as sodium, lithium and potassium salts, alkaline earth metal salts such as calcium and magnesium salts, salts with organic bases (for example, organic amines) such as dicyclohexylamine, abutyl amine, choline and salts with amino acids such as arginine, lysine and the like. Basic nitrogen containing groups may be quarternized with agents such as lower alkyl halides (e.g., methyl, ethyl and butyl chlorides, bromides and iodides), dialkyl sulfates (e.g., dimethyl, diethyl and dibutyl sulfates), long chain halides (e.g., decyl, lauryl and stearyl chlorides, bromides and iodides), aralkyl halides (e.g., benzyl and phenethyl bromides) and others. Compounds carrying an acidic moiety can be mixed with suitable pharmaceutically acceptable salts to provide, for example, alkali metal salts (e.g., sodium or potassium salts), alkaline earth metal salts (e.g., calcium or magnesium salts) and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

All such acid salts and base salts are intended to be pharmaceutically acceptable salts within the scope of the application and all acid and base salts are considered equivalent to the free forms of the corresponding compounds for purposes of the application. In addition, when a compound of the application contains both a basic moiety, such as, but not limited to an aliphatic primary, secondary, tertiary or cyclic amine, an aromatic or heteroaryl amine, pyridine or imidazole and an acidic moiety, such as, but not limited to tetrazole or carboxylic acid, zwitterions (“inner salts”) may be formed and are included within the terms “salt(s)” as used herein. It is understood that certain compounds of the application may exist in zwitterionic form, having both anionic and cationic centers within the same compound and a net neutral charge. Such zwitterions are included within the application.

Solvates of compounds of the application include, for example, those made with solvents that are pharmaceutically acceptable. Examples of such solvents include water (resulting solvate is called a hydrate) and ethanol and the like. Suitable solvents are physiologically tolerable at the dosage administered.

Prodrugs of the compounds of the present application include, for example, conventional esters formed with available hydroxy, thiol, amino or carboxyl groups. Some common esters which have been utilized as prodrugs are phenyl esters, aliphatic (C₁-C₂₄) esters, acyloxymethyl esters, carbamates and amino acid esters.

It is understood and appreciated that in some embodiments, compounds of the present application may have at least one chiral center and therefore can exist as enantiomers and/or diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

In some embodiments, the compounds of the present application can also include tautomeric forms, such as thioketo-enol tautomers and the like. Tautomeric forms can be in equilibrium or sterically locked into one form by appropriate substitution. It is intended that any tautomeric forms which the compounds form, as well as mixtures thereof, are included within the scope of the present application.

The compounds of the present application may further exist in varying amorphous and polymorphic forms and it is contemplated that any amorphous forms, polymorphs, or mixtures thereof, which form are included within the scope of the present application.

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier.

A compound of the application is suitably used on their own but will generally be administered in the form of a composition in which the one or more compounds of the application (the active ingredient) is in association with an acceptable carrier.

The compounds of the application may be administered to a subject in a variety of forms depending on the selected route of administration, as will be understood by those skilled in the art. One or more compounds of the application may be administered, for example, by oral, parenteral, buccal, sublingual, nasal, rectal, patch, pump or transdermal administration and the pharmaceutical compositions formulated accordingly. Administration can be by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists.

One or more compounds of the application may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound may be incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. Liposomes can be formed from a variety of phospholipids, such as cholesterol, stearylamine or phosphatidylcholines. For oral administration in a capsule form, useful carriers or diluents include lactose and dried corn starch.

Liquid preparations for oral administration may take the form of, for example, solutions, syrups or suspensions, or they are suitably presented as a dry product for constitution with water or other suitable vehicle before use. When aqueous suspensions and/or emulsions are administered orally, one or more compounds of the application are suitably suspended or dissolved in an oily phase that is combined with emulsifying and/or suspending agents. If desired, certain sweetening and/or flavoring and/or coloring agents may be added. Such liquid preparations for oral administration may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

One or more compounds of the application may also be administered parenterally. Solutions of the one or more compounds of the application can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

The compounds of the application may be formulated for parenteral administration by injection, including using conventional catheterization techniques or infusion. Formulations for injection may be presented in unit dosage form, e.g., in ampoules or in multi-dose containers, with an added preservative. The compositions may take such forms as sterile suspensions, solutions or emulsions in oily or aqueous vehicles, and may contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

Compositions for nasal administration may conveniently be formulated as aerosols, drops, gels and powders.

For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, PA, 1980, pp. 1530-1533 for further discussion of suppository dosage forms.

Compounds of the application may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, compounds of the application may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

In an embodiment, compounds of the application may be coupled with viral, non-viral or other vectors. Viral vectors may include retrovirus, lentivirus, adenovirus, herpesvirus, poxvirus, alphavirus, vaccinia virus or adeno-associated viruses. Non-viral vectors may include nanoparticles, cationic lipids, cationic polymers, metallic nanoparticles, nanorods, liposomes, micelles, microbubbles, cell-penetrating peptides, or lipospheres. Nanoparticles may include silica, lipid, carbohydrate, or other pharmaceutically acceptable polymers.

In some embodiments, depending on the mode of administration, the pharmaceutical composition will comprise from about 0.05 wt % to about 99 wt % or about 0.10 wt % to about 70 wt %, of the one or more compounds of the application, and from about 1 wt % to about 99.95 wt % or about 30 wt % to about 99.90 wt % of one or more pharmaceutically acceptable carriers, all percentages by weight being based on the total composition.

In some embodiments, the pharmaceutical composition is formulated for oral administration. In some embodiments, the compounds of the application are administered in a dose of about 0.01 mg/kg body weight to about 250 mg/kg body weight, about 0.1 mg/kg to about 230 mg/kg, about 1 mg/kg to about 210 mg/kg, about 30 mg/kg to about 200 mg/kg, about 50 mg/kg to about 180 mg/kg, about 60 mg/kg, to about 150 mg/kg, about 80 mg/kg to about 120 mg/kg once daily or twice daily.

In some embodiments, one or more compounds of the application are administered with another therapeutic agent simultaneously or sequentially in separate unit dosage forms or together in a single unit dosage form. Accordingly, the present application provides a single unit dosage form comprising one or more compounds of the application, an additional therapeutic agent, and a pharmaceutically acceptable carrier.

In some embodiments, the additional therapeutic agent is an antiviral agent. In some embodiments, the antiviral agent is selected from acyclovir, valacyclovir, famciclovir, brivudine, docosanol, idoxuridine, penciclovir or trifluridine, or combinations thereof. In some embodiments, the antiviral agent is acyclovir.

In the above, the term “a compound” also includes embodiments wherein one or more compounds are referenced.

### III. Method and Uses of the Application

Exemplary compounds of the application were tested in human neural progenitor cell (NPCs) lines and brain organoids and were shown to reduce the percentage of the reported genes EGFP+-RFP+, whose expression is under the control of viral promoters ICP0 and Glycoprotein C, respectively. In the neural precursor cell (NPCs) lines and in the brain organoids, exemplary compounds of the application showed anti-HSV-1 activity that was comparable to acyclovir and trans-dihydrolycoricidine (2b) or improved anti-HSV-1 activity. The anti-HSV-1 activity was also confirmed in a mouse model. Further, the exemplary compounds of the application showed improved anti-SARS-COV2 activity and improved therapeutic selectivity, compared to the 2b compound. Thus, the exemplary compounds of the application demonstrated potent broad-spectrum antiviral activity, in both RNA and DNA viruses. Further results demonstrated increased a subunit of eukaryotic initiation factor 2 (eIF2α) immunoreactivity in the cultures treated with the exemplary compounds of the application, suggesting that the exemplary compounds trigger a host antiviral response by activating an integrated stress response (ISR).

In some embodiments, the present application includes a use of a compound of the application as a medicament.

In some embodiments, the compound of the application is useful in the treatment of a disease, disorder or condition treatable by activating the integrated stress response via upregulation of eukaryotic initiation factor 2α (eIF2α). Accordingly, the present application also includes a method of treating a disease, disorder or condition treatable by activating the integrated stress response via upregulation of eukaryotic initiation factor 2α (eIF2α) comprising administering a therapeutically effective amount of a compound of the application to a subject in need thereof. The present application also includes a use of a compound of the application for treating a disease, disorder or condition treatable by activating the integrated stress response via upregulation of eukaryotic initiation factor 2α (eIF2α) as well as a use of a compound of the application for the preparation of a medicament for treating a disease, disorder or condition treatable by activating the integrated stress response via upregulation of eukaryotic initiation factor 2α (eIF2α) in a subject in need thereof. The application further includes a compound of the application for use in treating a disease, disorder or condition treatable by activating the integrated stress response via upregulation of eukaryotic initiation factor 2α (eIF2α) in a subject in need thereof.

In some embodiments, the compound of the application is useful in a method of activating the integrated stress response via upregulation of eukaryotic initiation factor 2α (eIF2α) in a cell, either in a biological sample or in a subject. Accordingly, the present application also includes a method of activating the integrated stress response via upregulation of eukaryotic initiation factor 2α (eIF2α) in a cell, either in a biological sample or in a subject comprising administering a therapeutically effective amount of one or more compounds of the application, to a cell in need thereof. The present application also includes a use of a compound of the application for activating the integrated stress response via upregulation of eukaryotic initiation factor 2α (eIF2α) in a cell, either in a biological sample or in a subject, as well as a use of a compound of the application for the preparation of a medicament for activating the integrated stress response via upregulation of eukaryotic initiation factor 2α (eIF2α) in a cell, either in a biological sample or in a subject. The application further includes a compound of the application for use in activating the integrated stress response via upregulation of eukaryotic initiation factor 2α (eIF2α) in a cell, either in a biological sample or in a subject.

In some embodiments, the compound of the application is useful in the treatment of a viral infection. Accordingly, the present application also includes a method of treating a viral infection comprising administering a therapeutically effective amount of a compound of the application to a subject in need thereof. The present application also includes a use of a compound of the application for treatment of a viral infection as well as of a use of a compound of the application for the preparation of a medicament for treatment of a viral infection in a subject in need thereof. The application further includes a compound of the application for use in treating a viral infection in a subject in need thereof.

In some embodiments, the viral infection comprises an infection with DNA or RNA viruses.

In some embodiments, the DNA virus is HSV-1.

In some embodiments, the RNA virus is SARSCoV-2.

In some embodiments, the subject is a mammal. In some embodiments, the subject is a human. In some embodiments, the subject is a non-human animal. Accordingly, the compounds, methods and uses of the present application are directed to both human and veterinary diseases, disorders and conditions.

In some embodiments, the one or more compounds of the application are administered with one or more additional therapeutic agents simultaneously or sequentially in separate unit dosage forms or together in a single unit dosage form. Accordingly, the present application provides a single unit dosage form comprising one or more compounds of the application one or more additional therapeutic agents, and a pharmaceutically acceptable carrier. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art.

In some embodiments, the one or more additional therapeutic agents is an antiviral agent. In some embodiments, the antiviral agent is acyclovir.

Effective amounts may vary according to factors such as the disease state, age, sex and/or weight of the subject. The amount of a given compound that will correspond to such an amount will vary depending upon various factors, such as the given drug or compound, the pharmaceutical formulation, the route of administration, the type of condition, disease or disorder, the identity of the subject being treated, and the like, but can nevertheless be routinely determined by one skilled in the art. The effective amount is one that following treatment therewith manifests as an improvement in or reduction of any disease symptom.

In some embodiments, a compound of the application is administered by oral, parenteral, buccal, sublingual, nasal, rectal, patch, pump or transdermal administration. In some embodiments, a compound of the application is administered by ocular, topical, transdermal or oral administration or by inhalation or injection.

The dosage of a compound of the application can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. Compounds of the application may be administered initially in a suitable dosage that may be adjusted as required, depending on the clinical response. Dosages will generally be selected to maintain a serum level of compounds from about 0.01 μg/cc to about 1000 μg/cc, or about 0.1 μg/cc to about 100 μg/cc. As a representative example, oral dosages of a compound of the application will range between about 1 mg per day to about 1000 mg per day for an adult, suitably about 10 mg, about 25 mg, about 50 mg, about 100 mg, about 150 mg, about 200 mg, about 250 mg, about 300 mg, about 350 mg, about 400 mg, about 450 mg, about 500 mg, about 550 mg, about 600 mg or about 650 mg of per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg or about 0.1 mg/kg to about 10 mg/kg. For administration in suppository form, a representative amount is from about 0.1 mg/kg to about 10 mg/kg. In some embodiments, of the application, compositions are formulated for oral administration and the compounds are suitably in the form of tablets or suppositories containing 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient per tablet. Compounds of the application may be administered in a single daily, weekly or monthly dose or the total daily dose may be divided into two, three, four, five or six daily doses.

In some embodiments, the pharmaceutical composition is formulated for oral administration. In some embodiments, the pharmaceutical composition is formulated for rectal administration. In some embodiments, the one or more compounds of the application are administered in a dose of about 0.01 mg/kg body weight to about 250 mg/kg body weight, about 0.1 mg/kg to about 230 mg/kg, about 1 mg/kg to about 210 mg/kg, about 30 mg/kg to about 200 mg/kg, about 50 mg/kg to about 180 mg/kg, about 60 mg/kg, to about 150 mg/kg, about 80 mg/kg to about 120 mg/kg and values therebetween in a single daily, weekly or monthly dose or the total daily dose may be divided into two, three, four, five or six daily doses.

In some embodiments, one or more compounds of the application are administered at least once a week. However, in another embodiment, one or more compounds of the application are administered to the subject from about one time per two weeks, three weeks or one month. In another embodiment, one or more compounds of the application are administered about one time per week to about once daily. In another embodiment, one or more compounds of the application are administered 2, 3, 4, 5 or 6 times daily. The length of the treatment period depends on a variety of factors, such as the severity of the disease, disorder or condition, the age of the subject, the concentration and/or the activity of a compound of the application, and/or a combination thereof. It will also be appreciated that the effective dosage of one or more compounds of the application are used for the treatment may increase or decrease over the course of a particular treatment regime. Changes in dosage may result and become apparent by standard diagnostic assays known in the art. In some instances, chronic administration is required. For example, the compounds are administered to the subject in an amount and for duration sufficient to treat the subject.

All references to “a compound” above, also include embodiments where one or more compounds are administered or used.

### IV. Methods of Preparing the Compounds of the Application

Compounds of the application can be prepared by various standard chemistries known in the art. The choice of particular structural features and/or substituents may influence the selection of one process over another. The selection of a particular process to prepare a given compound of the application is within the purview of the person of skill in the art. Some starting materials for preparing compounds of the application are available from commercial chemical sources. Other starting materials, for example as described below, are readily prepared from available precursors using straightforward transformations that are well known in the art. In the Schemes below showing the preparation of compounds of the application, all variables are as defined in Formula I, unless otherwise stated.

In some embodiments, where R² is hydrogen, a compound of Formula I may be prepared as shown in Schemes 1-2, where R¹, X¹ and X² are as defined in Formula I.

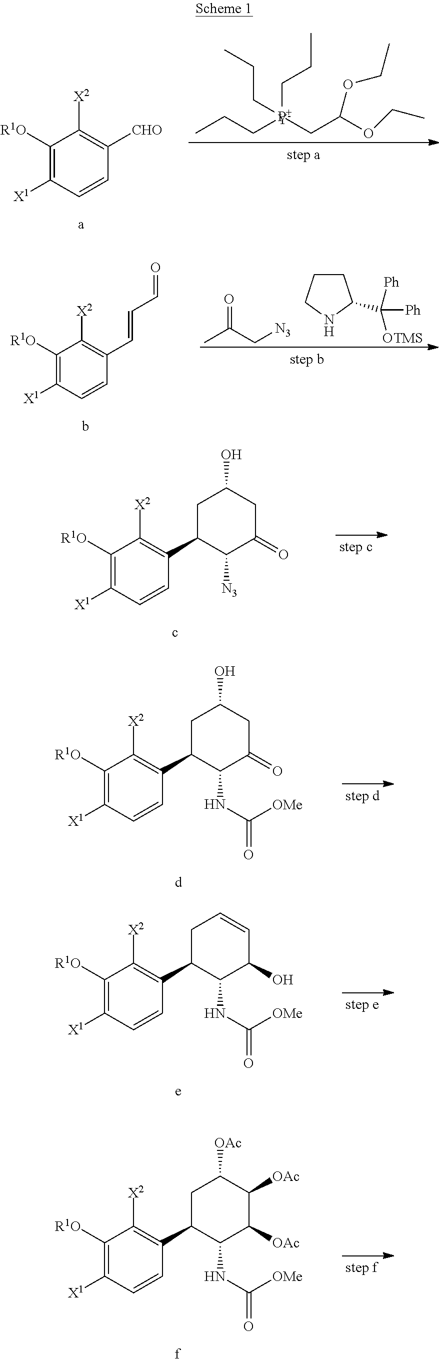

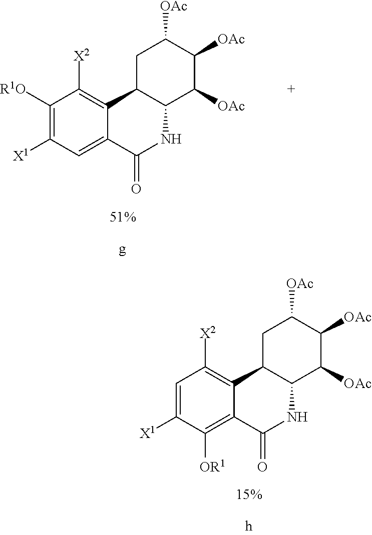

In step a, benzaldehyde compound a is subject to two-carbon aldehyde to alkenal homologation using an acetal-functionalized Wittig reagent (such as tripropyl-(2,2-diethoxyethyl)-phosphonium bromide) with a suitable base and solvent (such as NaH base and THE solvent), followed by a treatment with a suitable acid (such as HCl) to yield a cinnamaldehyde b.

In step b, compound b may react with azidoacetone in a suitable solvent (such as DCM) in the presence of a suitable one or more catalysts (such as (R)-diphenylprolinol-trimethylsilyl ether and quinidine catalysts) to produce compound c.

In step c, compound c may react in the presence of a suitable catalyst (such as Pd) and solvent (such as MeOH) to produced the methoxycarbonyl protected cyclohexanone d.

In step d, compound d may be treated with a dehydrating agent (such as MsCl), using a suitable base (such as DIPEA) and solvent (such as DCM), followed by a reduction with a suitable agent (such as lithium tri-tert-butoxyaluminium hydride) in a suitable solvent (such as THF) to obtain compound e.

Epoxidation of compound e, in step e, in the presence of a suitable oxidizing reagent (such as mCPBA), a base (such as sodium bicarbonate) and a solvent (such as DCM), followed by treatment with a suitable nucleophile (such as sodium benzoate), and then acetylating agent (such as Ac₂O) produces compound f.

The phenanthridone ring closure in step f, using suitable electrophilic reagent (such as Tf₂O), base (such as DMAP) and a solvent (such as DCM) produces two compounds g and h. Compound h is used in the next steps to produce the exemplary compounds of the application.

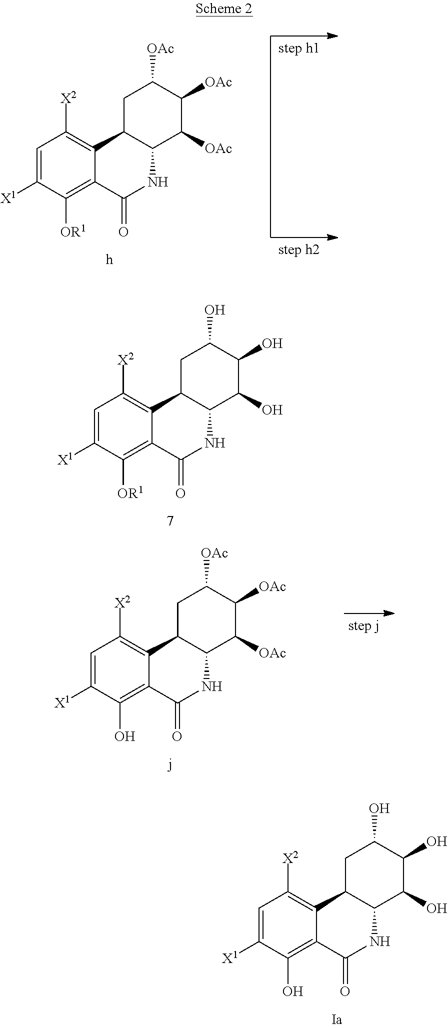

In the next step (h1), compound h is hydrolyzed with a suitable acid (K₂CO₃) and solvent (MeOH) to obtain exemplary compound 7. To obtain the exemplary compound Ia, compound h may be treated in step h2 with a suitable acid (such as AlCl₃) in the presence of a catalyst (such as tetrabutylammonium iodide) and a solvent (such as DCM), followed by hydrolysis with a suitable acid (K₂CO₃) and solvent (MeOH).

Salts of the compound of Formula I, or a solvate thereof, are generally formed by dissolving the neutral compound in an inert organic solvent and adding either the desired acid or base and isolating the resulting salt by either filtration or other known means.

The formation of solvates of the compound of Formula I, or a salt thereof, will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

The products of the processes of the application may be isolated according to known methods, for example, the compounds may be isolated by evaporation of the solvent, by filtration, centrifugation, and/or chromatography or other suitable method.

One skilled in the art will recognize that where a reaction step of the present application is carried out in a variety of solvents or solvent systems, said reaction step may also be carried out in a mixture of the suitable solvents or solvent systems.

### EXAMPLES

The following non-limiting examples are illustrative of the present disclosure:

### Results and Discussion

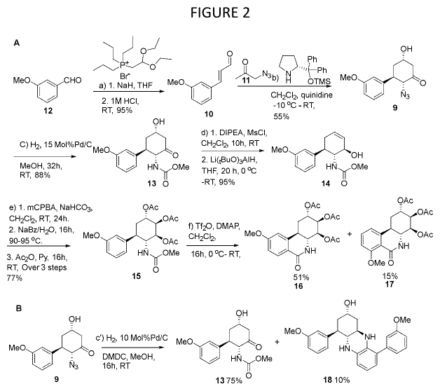

Given the anticancer activity demonstrated by certain members of the lycorane class,² the development of a viable antiviral compound requires careful analysis of the antiviral structure-activity and monitoring of the cytotoxicity profile in order to navigate a selective compound. In addition to the total synthesis and antiviral studies of 1-deoxypancratistatin (2a ) and trans-dihydrolycoricidine (2b),^{4a,b} the synthesis of various modifications to the hydroxylated ring-C and their antiviral activities was investigated. For example, the C3-epimer of 2b,^{5a} among other analogs, as well as the fully functionalized aza-analog 4^{5b} proved totally devoid of antiviral activity. To date, compound 2b appears to be the minimal structure that exhibits potent antiviral activity, activity that is significantly enhanced by addition of the C7 hydroxyl group present in compound 2a.^{3a} Further modifications to ring-C as a means of optimizing antiviral potency and selectivity further appear fruitless. These results turned the focus to the ring-A substituents as a fragment available for modification. All analogs investigated for RNA viral activity possess the 8,9-methylenedioxy substituent fused to ring-A.^{3f} Only one prior study has investigated alternative ring-A hydroxyl positionings. Alonso and co-worker reported the synthesis of the 8-hydroxyl analog, which was found to exhibit significantly reduced anticancer activity, while antiviral activity was not investigated.⁶ In order to delineate the contributions of a single hydroxyl or methoxy group substituent in ring-A to antiviral activity, the original synthesis of 1-deoxypancratistatin (2a ) and trans-dihydrolycoricidine (2b) was modified, starting from meta-anisaldehyde (12) (FIG. 2).

The synthesis of the target compounds 5, 6, 7 and Ia was achieved by a convergent-divergent strategy via the common intermediate 15 (FIG. 2, A) with significant modifications to previously published synthetic protocol.⁴ Commercially available 3-methoxybenzaldehyde 12 (FIG. 2) was subject to a two-carbon aldehyde to alkenal homologation using a diethyl acetal-functionalized Wittig reagent yielding the 3-methoxy cinnamaldehyde 10 in 95% yield. The iminium-ion mediated asymmetric organocatalytic stepwise [3+3] Michael-aldol sequence of 10 with azidoacetone 11 proceeded smoothly using the (R)-diphenylprolinol-trimethylsilyl ether and quinidine catalysts, providing the cycloadduct 9 in 55% isolated yield. Hydrogenation of the azido functional group of 9 with 10% Pd/C in the presence of dimethyldicarbonate gave the desired methoxycarbonyl protected cyclohexanone 13 as well as a minor side-product, which proved to be the structurally interesting mono-aromatized dimer 18 (FIG. 2, B). To improve the selectivity favouring 13, dilution (0.1 M) and increased reaction time allowed isolation of the methoxycarbonyl protected cyclohexanone 13 in 75% isolated yield along with the minor product 18 in 10% isolated yield. Interestingly, increased catalyst loading up to 15%, under otherwise identical reaction conditions, led only to the desired compound 13. This minor side product indicates that the anticipated reduction of the azide 9 was successful, the free amino-ketone then undergoes an in-situ dimerization followed by dehydration and aromatization of one ring (perhaps via an enamine intermediate) producing 18. The carbamate 13 and its enantiomer ent-13, prepared identically using (S)-diphenylprolinol-trimethylsilyl ether) were both found to be >99% and >98% enantiomeric excess (e.e.) respectively (baseline resolved) using chiral HPLC (AD-H column). Dehydration of 13 via the in-situ formed mesylate and immediate reduction of the ketone using the bulky lithium tri-tert-butoxyaluminium hydride yielded the equatorial alcohol 14 exclusively in 95% yield for the two steps. Epoxidation of the allylic alcohol 14 produced both diastereomeric epoxide which are opened stereospecifically through exclusive axial attack using sodium benzoate in water, yielding the 2,3-diaxial-4-equatorial triol, which was acetylated with Ac₂O in the presence pyridine to give triacetate 15 in 77% overall yield from 13. The phenanthridone ring was closed employing Banwell's modification of the Bischler-Napieralski reaction,⁷ giving the regioisomeric tricyclic products 16 and 17. Compounds 16 and 17 were clearly resolved on silica and separated by gradient elution flash chromatography starting from 100% dichloromethane, yielding 16 (major) and 17 (minor) in 51% and 15% isolated yields respectively.

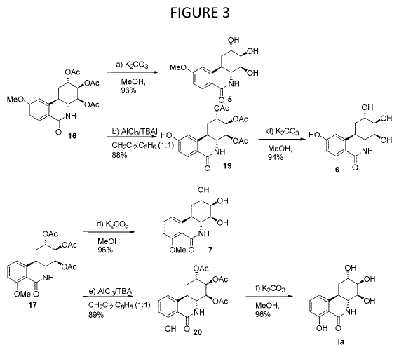

Selective cleavage of the C9-methyl ether in 16 proved problematic. Attempted cleavage using standard reagents such as TMSI or BBr₃ gave mixtures of products, and the use of BCl₃-dimethylsulfide resulted in ester cleavage. Nevertheless, it was discovered that the use of AlCl₃ in the presence of the phase transfer catalyst tetrabutylammonium iodide (1:3) was successful, following a protocol developed by Steglich and co-workers.⁸ This procedure provided phenolic compound 19 in 88% yield (FIG. 3). The same reaction conditions were repeated for the cleavage of C7-methyl ether 17, which also provided the demethylated phenol 20 in 89% yield. Global saponification of the acetate groups was readily achieved in compounds 16, 19, 17 and 20 using K₂CO₃ and aqueous methanol finally yielding the required compounds 5, 6, 7, and Ia respectively (FIG. 3).

It was recently shown that trans-dihydrolycoricidine (2b) inhibits HSV-1 replication more efficaciously than acyclovir (ACV), and prevents HSV-1 reactivation from latency in an ex vivo mouse model.^{3d,5a} Also, 2b was shown to exhibit potency against HSV-1 strains that have been reported to be resistant to ACV, such as the tk-strain of HSV-1 that lacks thymidine kinase activity,⁹ and the PAAv strain that has developed mutations in viral DNA polymerase following incubation with phosphonoacetic acid.¹⁰ The antiviral activity of 2b against other DNA viruses (HSV-1, HCMV, HBV, HCV), and RNA viruses (ZIKV strains FSS-13025 and PE-243) was also demonstrated.^{3d} Slightly higher toxicity was observed for 2b in comparison to acyclovir in fibroblasts and hepatocytes, while a lower toxicity was noted in iPSC-derived neural progenitor cells (NPCs).

### C7-Monohydroxyl Analog Ia Exhibits Anti-HSV-1 Activity.

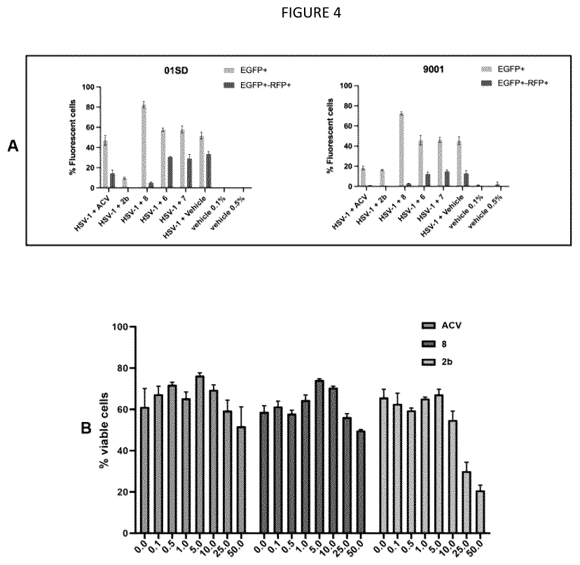

The antiviral activity of the new mono-hydroxyl and methoxy derivatives 5, 6, 7, and Ia derivatives was initially tested in two neural precursor cell (NPCs) lines and compared with acyclovir, the “gold standard” treatment for HSV infections. NPCs were utilized in this initial screen for two reasons; i) the need to employ a platform composed of CNS cells, and ii) the higher susceptibility of NPCs to HSV-1 when compared to neurons. Human NPCs were derived from iPSCs as previously described.¹¹ NPCs were infected with an HSV-1 construct (DualF) expressing enhanced green fluorescent protein (EGFP) and monomeric red fluorescent protein (RFP) under the control of the viral promoters ICP0 and Glycoprotein C, respectively,¹¹ at a multiplicity of infection (MOI) of 0.1, as detailed in the Experimental section B. Two hours after the infection the inocula were removed and cells were cultured in the presence of the above described monomethoxy/hydroxy analogs at the concentration of 10 μM or ACV at 50 μM, and assayed at day 3 post infection (p.i.). Flow cytometry (FC) analysis indicated that compound Ia reduced the percentage of EGFP+-RFP+ cells by 6.76- and 4.65-fold and in 01SD and 9001 NPCs, respectively (FIG. 4A). Compound 2b resulted in the greatest reduction of fluorescent cells (01SD: (8016-fold; 9001:424-fold). The antiviral efficacy of ACV infection resulted inferior to Ia in 01SD lines (p=0.0208) or comparable (p=0.3597) in 9001. Surprisingly, no anti-HSV-1 activity was exhibited by analogs 5, 6 or 7 (FIG. 4A). Compound 5 proved incompletely soluble in DMSO, and was therefore not considered further.

At concentrations up to 50 μM, the cell viability of ACV and compound Ia treated cultures were comparable (FIG. 4B). Conversely, a reduction in cell viability was observed in 2b-treated cultures starting from 25 μM in accord with the cytotoxicity previously discussed for 2b. Once again, no anti-HSV-1 activity was exhibited by compounds 6 and 7 (FIG. 4B). Thus, quantification of viral infection and cell viability based structural-activity relationship analysis using monolayer 2D NPC cultures identified compound Ia as a novel anti-HSV-1 drug with an efficacy comparable or superior to ACV.

### The Anti-HSV-1 Activity of Ia is Comparable to 2b in Brain Organoids.

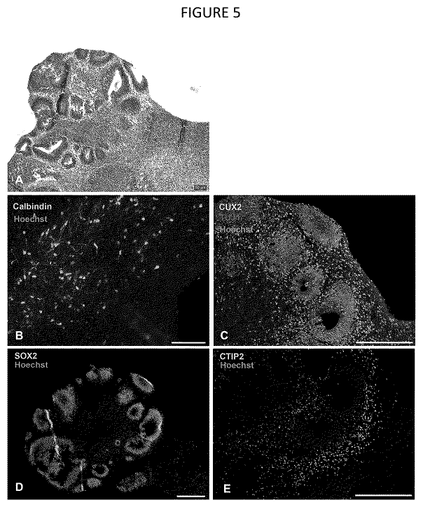

Although the classical 2D monolayer cultures facilitate drug screening processes, they do not recapitulate the 3D architecture and the physiological microenvironment of a living tissue conditions. As a consequence, drug effects in a 2D microenvironment may not accurately reflect drug effects in vivo. There is increasing evidence that three-dimensional cultures increase the predictability of drug activity in vivo.¹² Thus, the inhibitory activity of 2b and its derivatives against HSV-1 was investigated in a 3D culture platform consisting of brain organoids. A characterization of differentiating organoids is depicted in FIG. 5.

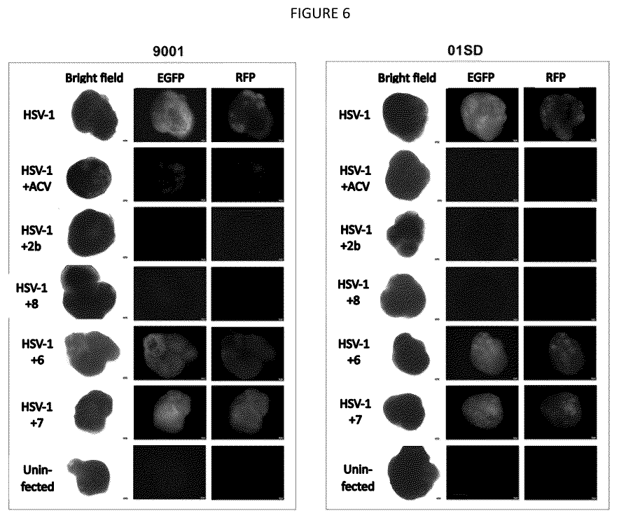

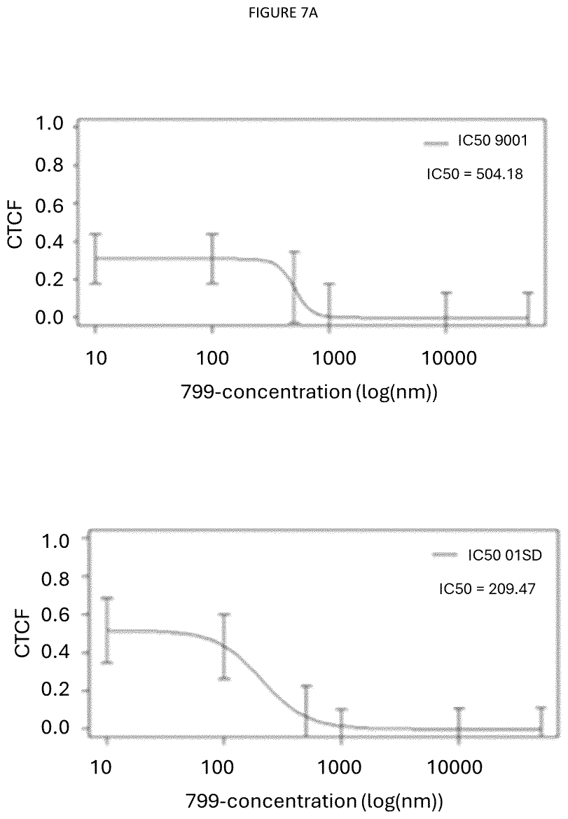

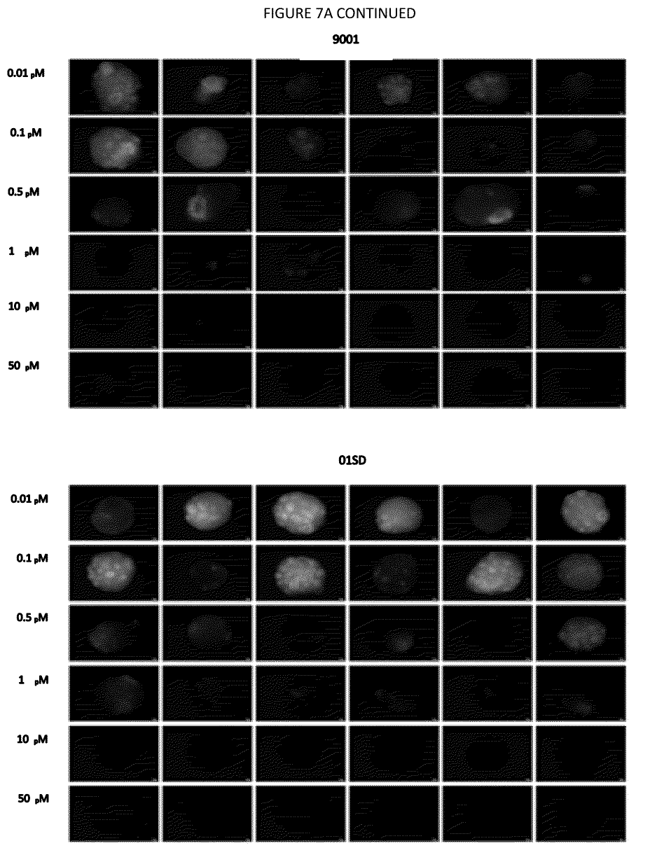

Fourteen-week old brain organoids generated from 9001 and 01SD iPSCs were infected singularly for 2 hours with HSV-1 DualF construct (6000 pfu/organoid). After the infection the organoids were cultured in the presence or absence of tested compounds (10 μM) or ACV (50 μM). At day 3 p.i. no expression of EGFP and RFP reporter genes was observed in 2b- and Ia-treated infected 9001 and 01SD organoids. As expected, ACV efficiently inhibited HSV-1 infection (FIG. 6). Conversely, no anti-HSV-1 activity was exhibited by derivatives 6 and 7 (FIG. 6). These results confirm the ability of the new analog Ia alone to inhibit HSV-1 infection.

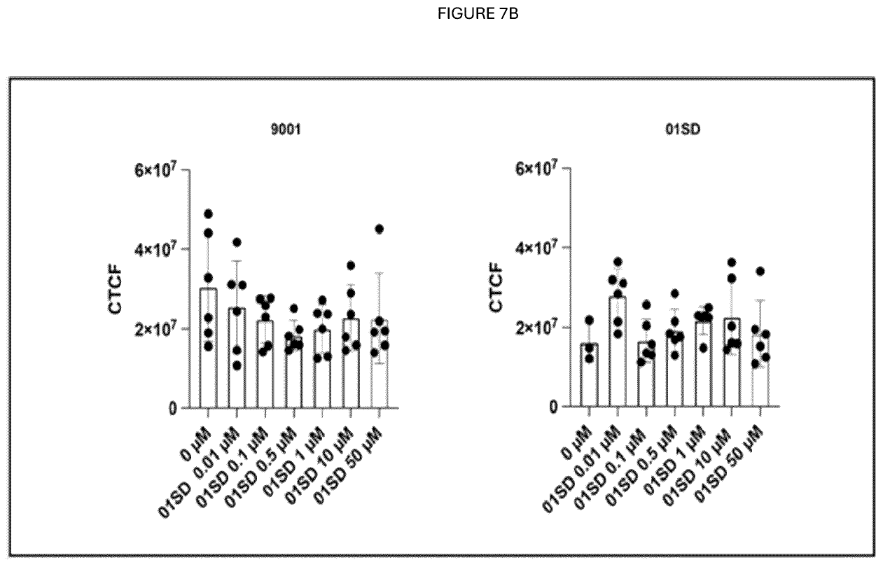

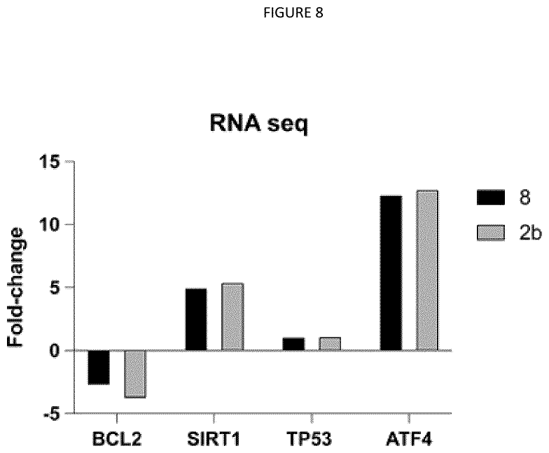

Next, the concentration of Ia that inhibited HSV-1 infection by 50% (IC50) in 9001 and 01SD organoids was determined. Sixteen-week old brain organoids were infected as described above and exposed after 2 hours to increasing Ia concentration ranging from 0.01-50 μM. At day 3 p.i. the IC50 of Ia was determined by measuring the intensity of the fluorescent reporter gene EGFP (whose expression is under the control of promoter of the immediate early HSV-1 gene ICP0) in the infected organoids was investigated. The EGFP expression was used as a proxy of viral gene expression during early stages of HSV-1 acute infection. The IC50 was determined to be 0.504 μM in 9001 and 0.209 μM in 01SD organoids (FIG. 7A). No significant reduction in organoid viability, determined by utilizing calcein AM, was observed at concentrations up to 50 μM (FIG. 7B).

### Compounds 2b and Ia Exhibit Potent Antiviral Activity in the NIH-NIAID in Vitro SARS-CoV2 Viral Replication Assay.

The antiviral activity of compounds 2b and Ia to the COVID-19 associated coronavirus SARS-CoV2 in Calu3 (ATCC, HTB-55) cells was assessed independently at the National Institutes of Health (NIH). Compound 2b was determined to exhibit an IC50 of 0.18 μM and CC of 3.48 μM, giving a therapeutic selectivity of 19. The new analog Ia proved to be both more potent and slightly more selective, exhibiting an IC50 of 0.09 μM and CC of 1.92 μM, giving a therapeutic selectivity of 20. The positive control remdesivir exhibited an IC50 value of 0.06 μM in this assay.

### RNA-Seq Analysis Demonstrates Compounds 2b and Ia Activate an Integrated Stress Response.

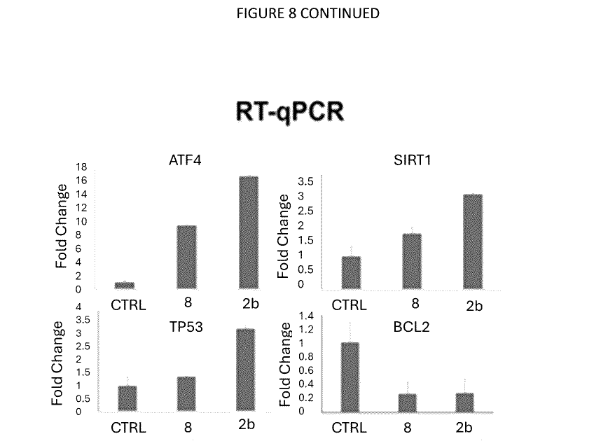

Given the activity observed to both DNA and RNA viruses with the synthetic compounds, RNA sequencing analysis was performed on host cells treated with the alkaloids in order to assess changes in transcription levels in the hope of illuminating operative pathways. Uninfected 02SF NPCs cells were incubated with 2b, Ia, 6 (802), or 7 (718), (10 μM) for 72 hours. Following extraction, host RNA sequences were analyzed. A total of 12,433 human genes were expressed at TPM>=5 in at least one replicate of untreated NPCs. When NPCs were incubated with com-pounds 2b, Ia, 6, or 7, the expression of 7,766 (62.5%), 7,433 (59.8%), 256 (2.1%), and 33 (0.3%) genes, respectively, were significantly altered (FDR corrected p<=0.05, maximum group mean expression TPM>=5). Using quantitative RT-PCR, alterations in mRNA levels for four selected genes that were significantly altered by compounds 2b and Ia (compound 8) in the RNA sequencing analyses were confirmed (FIG. 8).

Ingenuity Pathway Analysis (IPA) identified canonical pathways that were significantly altered when hiPSC-neurons were incubated with 2b and Ia.

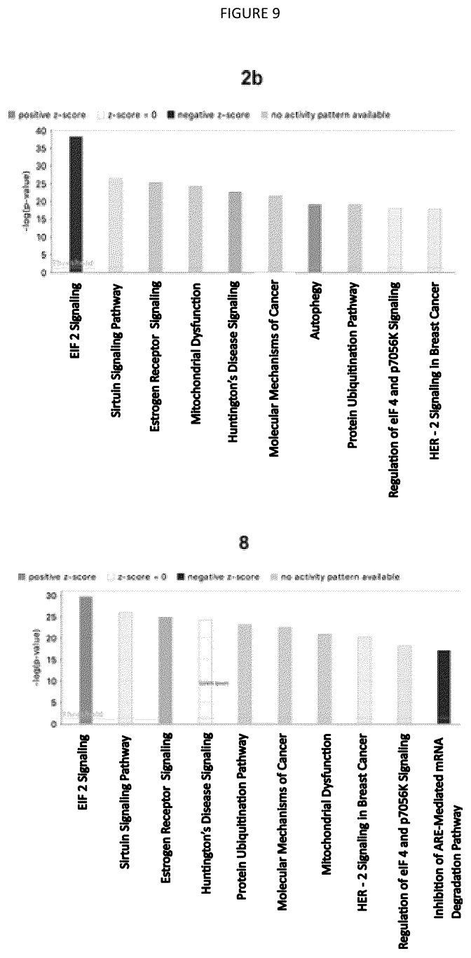

Statistical significance was calculated using right-tailed Fisher Exact Probability Tests; biological pathways showing p-value<0.05 were considered statistically significant. The ten most significantly altered pathways are shown in FIG. 9. The eukaryotic initiation factor 2 (EIF2) signaling pathway was most significantly altered (2b: z score:−2.857, p-value=5.09E-39; Ia: z score:−1.763, p-value=2.43E-30). IPA analyses also indicated net upregulation in the autophagy and sirtuin signaling pathways.

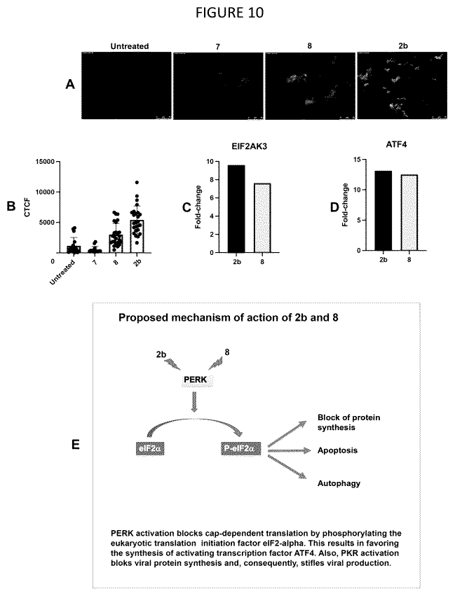

These results immediately suggested the hypothesis that compounds 2b and Ia mainly affect viral replication by triggering the integrated stress response, which, in turn, will cause a block in cap-dependent translation mRNA translation.^{13,14} To test this hypothesis, the phosphorylation of the a subunit of eukaryotic initiation factor 2 (eIF2α˜P) on serine 51 in NPC cultures exposed to compounds 2b and Ia was investigated, and compared to untreated NPCs or NPCs treated with the derivative 7, that does not exhibit any antiviral activity. Immunocytochemistry analysis indeed demonstrated increased eIF2α˜P immunoreactivity in the 2b- and Ia-treated cultures (FIG. 10). These results are fully in accord with the hypothesis that phosphorylation of e1F2α is upregulated and that activation of the integrated stress response (ISR) is one of the main mechanisms underlying the antiviral activity of these small molecules. RNAseq analysis suggested that phosphorylation of the a subunit of eukaryotic initiation factor 2 is mediated by the eIF2α kinase EIF2AK3 in the 2b- and Ia-treated cultures (FIG. 10).

ISR acts at the level of translation initiation to suppress global protein synthesis (while enhancing translation of several proteins involved in cellular recovery). ISR-induced downregulation of virus protein synthesis is an effective antiviral defense mechanism. Furthermore, eIF2-phosphorylation leads to increased synthesis of activating transcription factor 4 (ATF4), which then translocates to the nucleus to transactivate genes necessary for the cell to adapt the stress such as autophagy.¹⁵ In HSV-1-infected cells autophagy proteins target viral components or virions for lysosomal degradation.¹⁶ Thus, the RNA sequencing data thus suggest that 2b and Ia trigger a host antiviral response by activating an ISR. Furthermore, the activation of the sirtuin pathway reinforces the antiviral properties. Interestingly, SIRT1, a NAD-dependent deacylases/mono-ADP ribosyltransferase that can inhibit the growth of DNA and RNA viruses was shown to be up-regulated. The upregulation of SIRT1 may also contribute to the broad-spectrum antiviral activity observed with compounds 2b and Ia.^{17,18}

### Compound Ia Exhibits in Vivo Anti HSV-1 Activity in a Mouse Model.

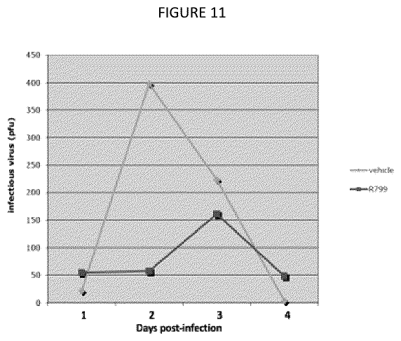

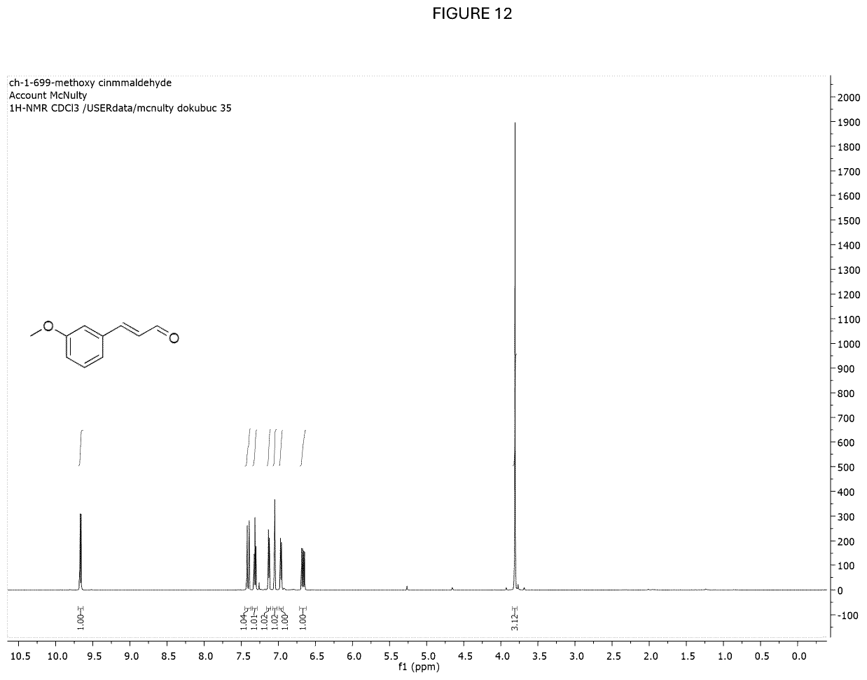

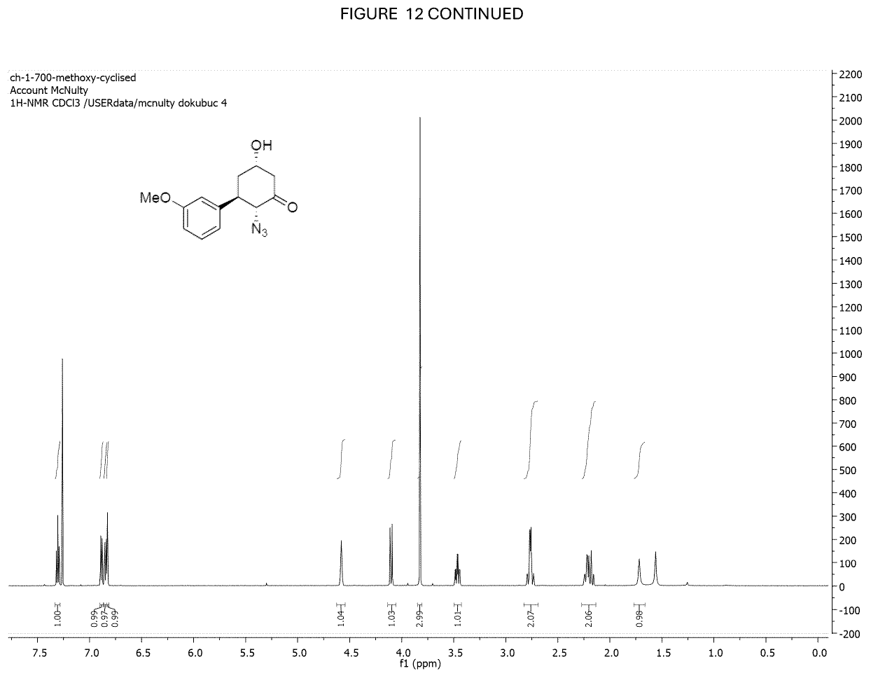

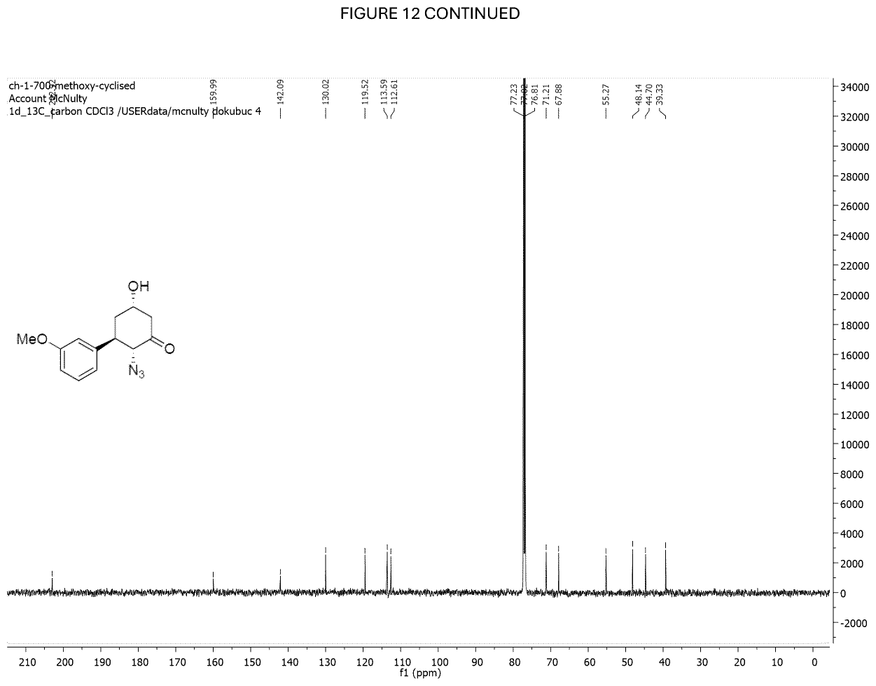

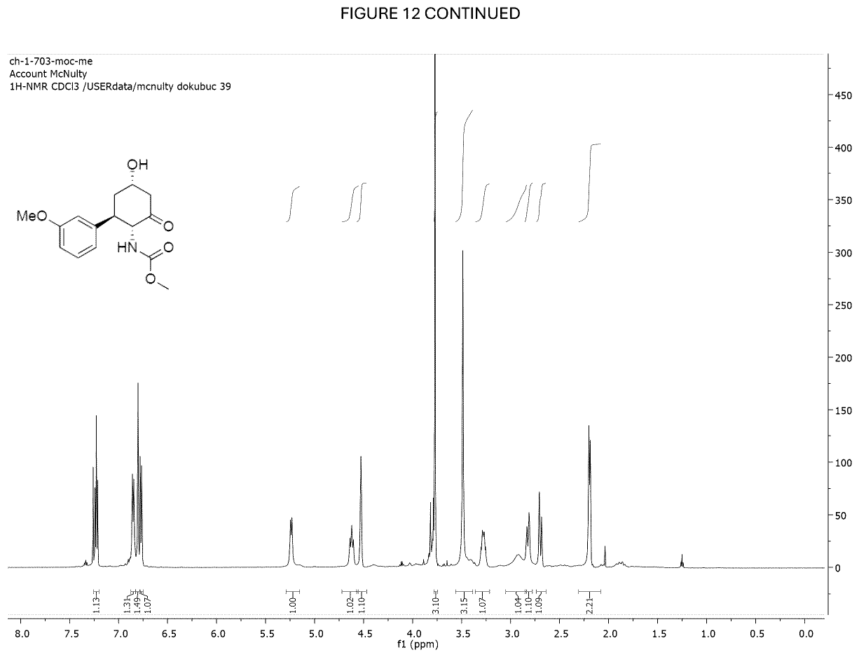

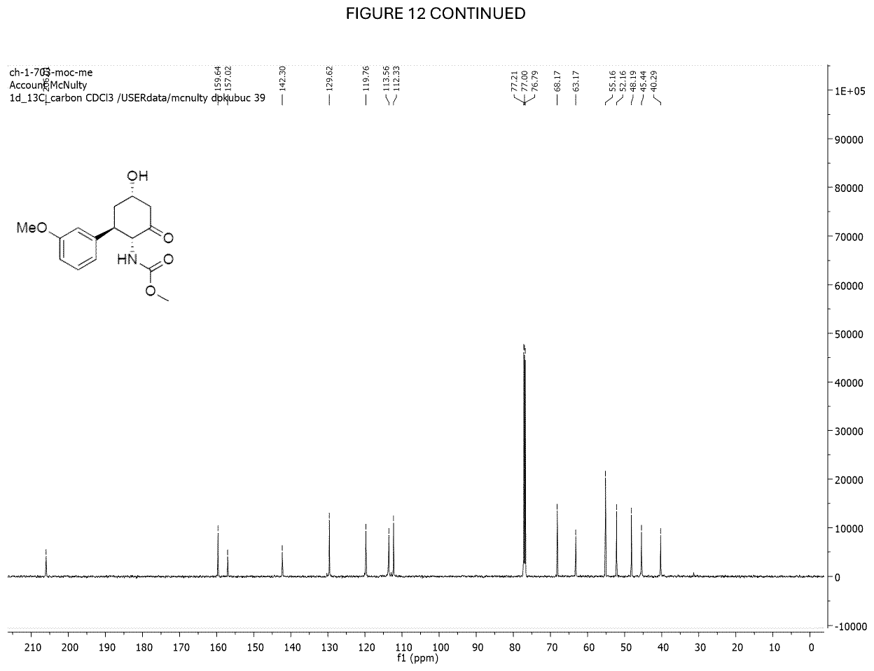

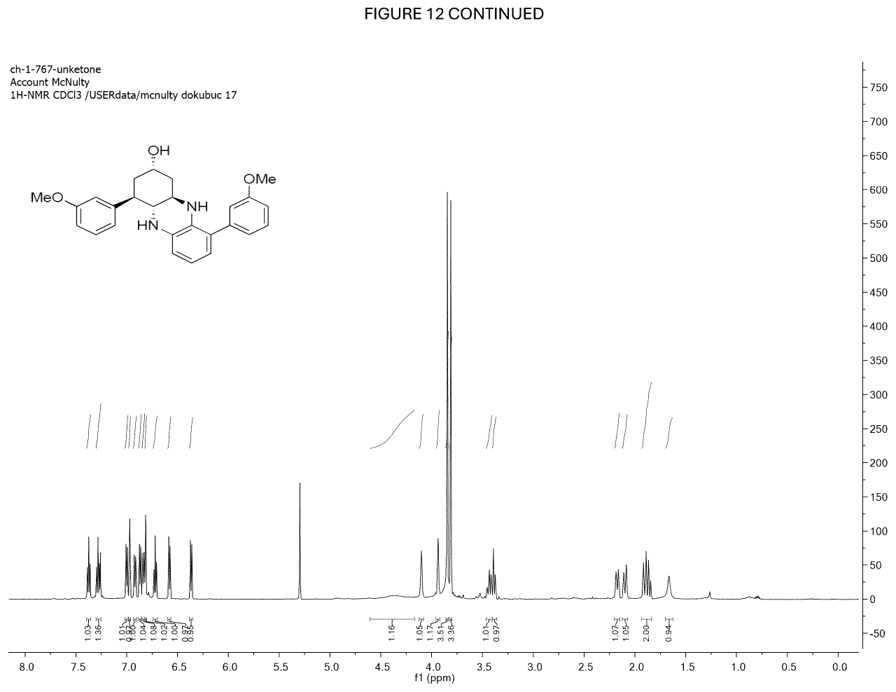

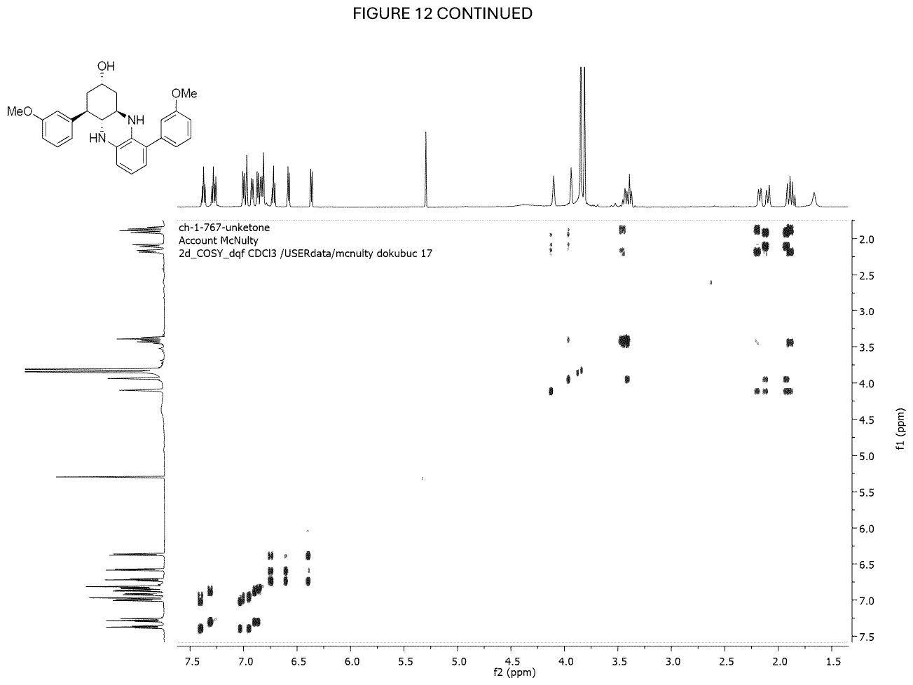

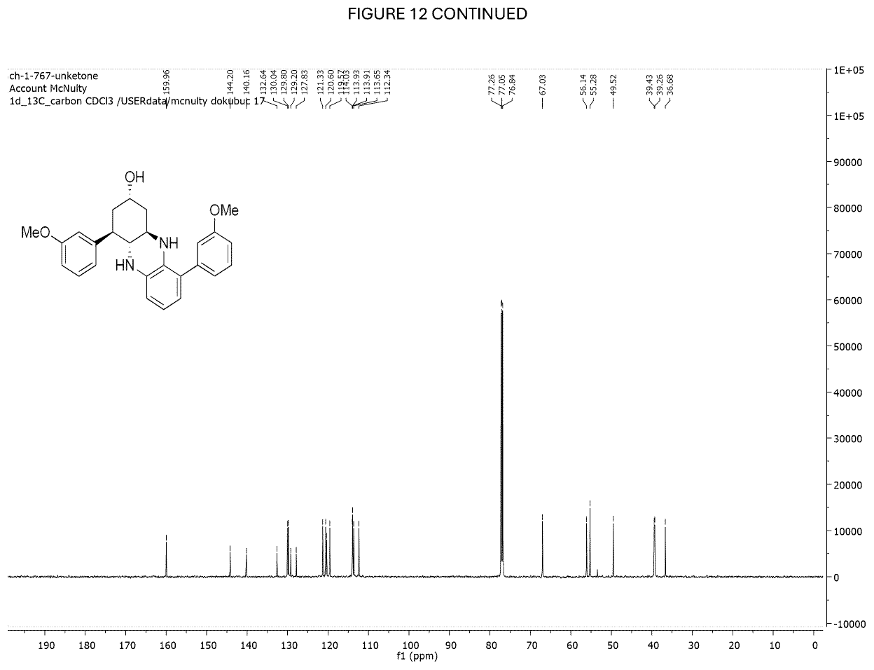

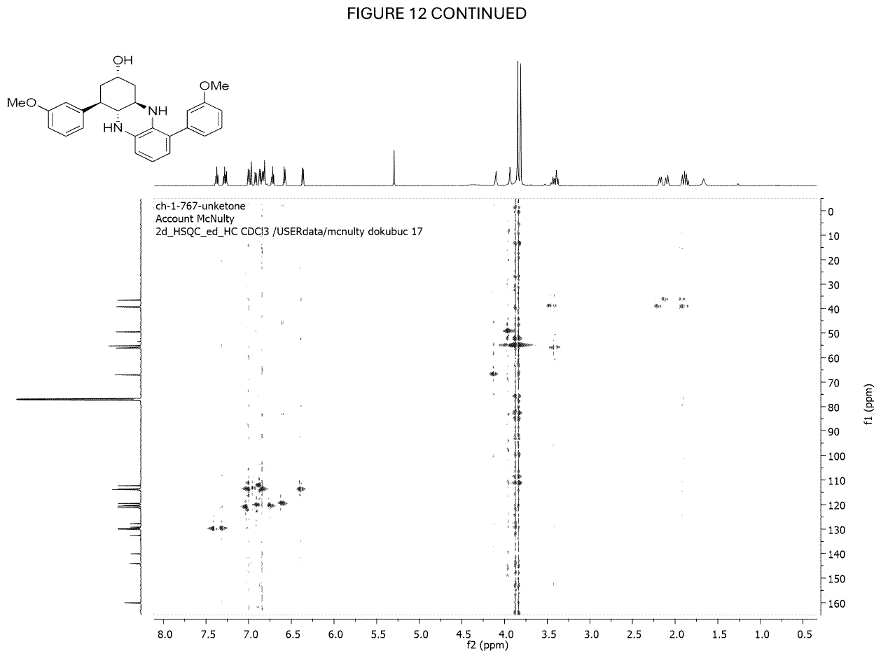

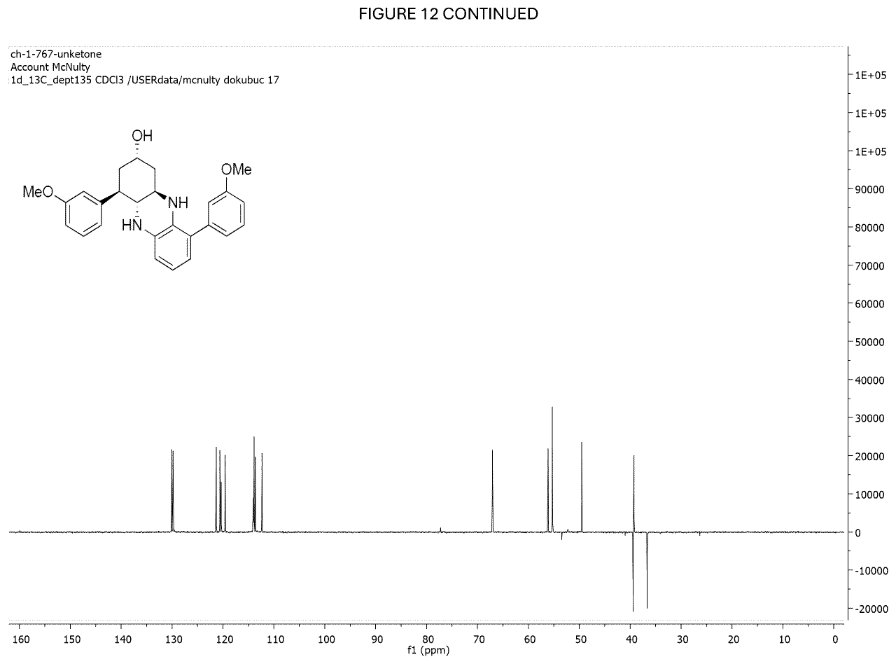

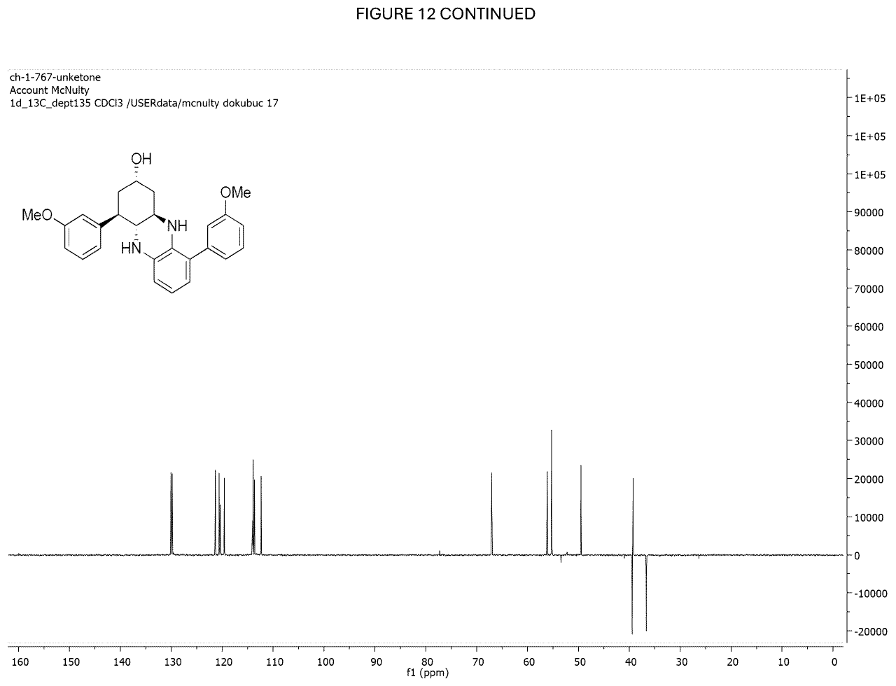

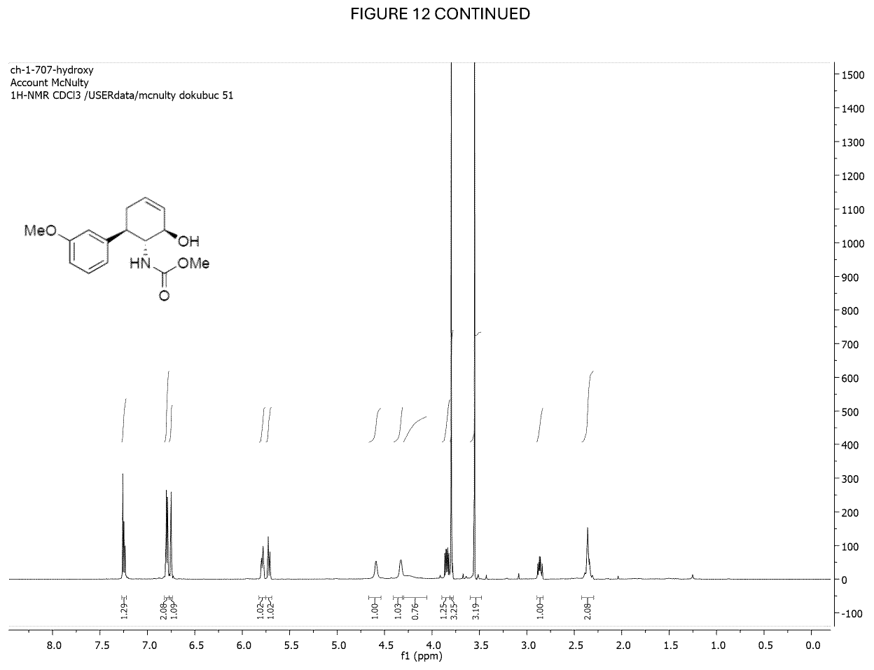

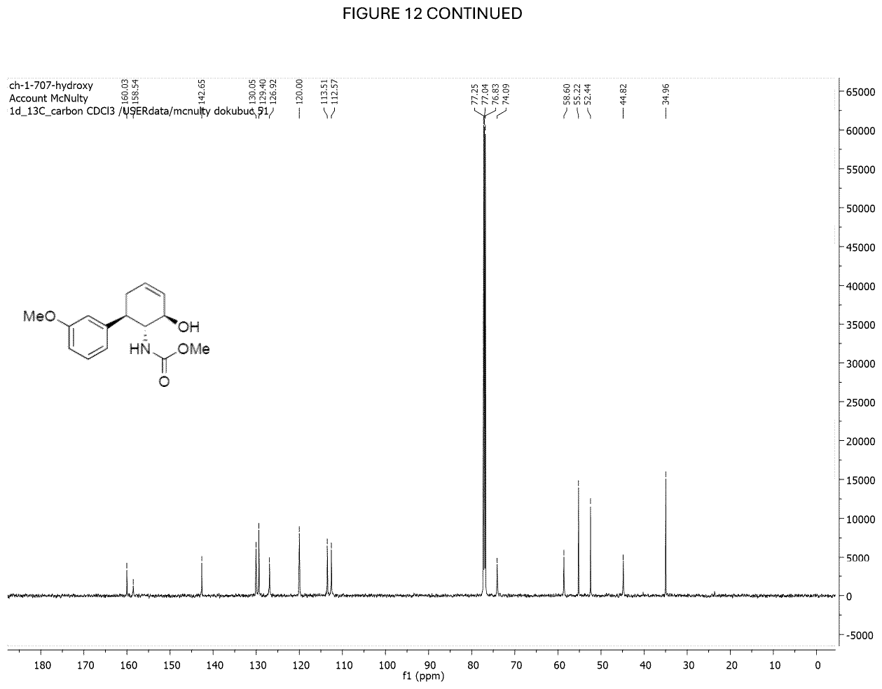

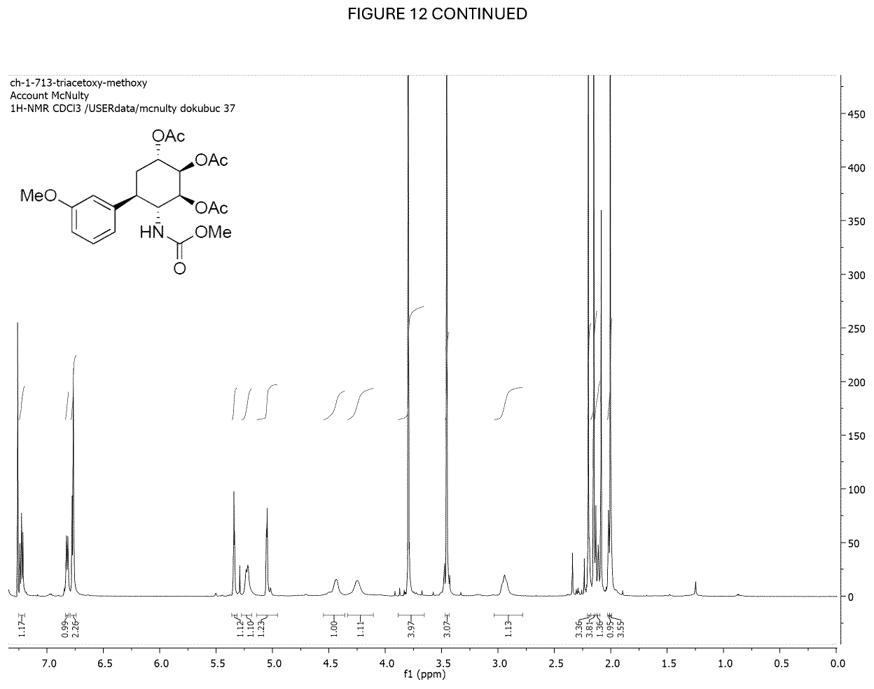

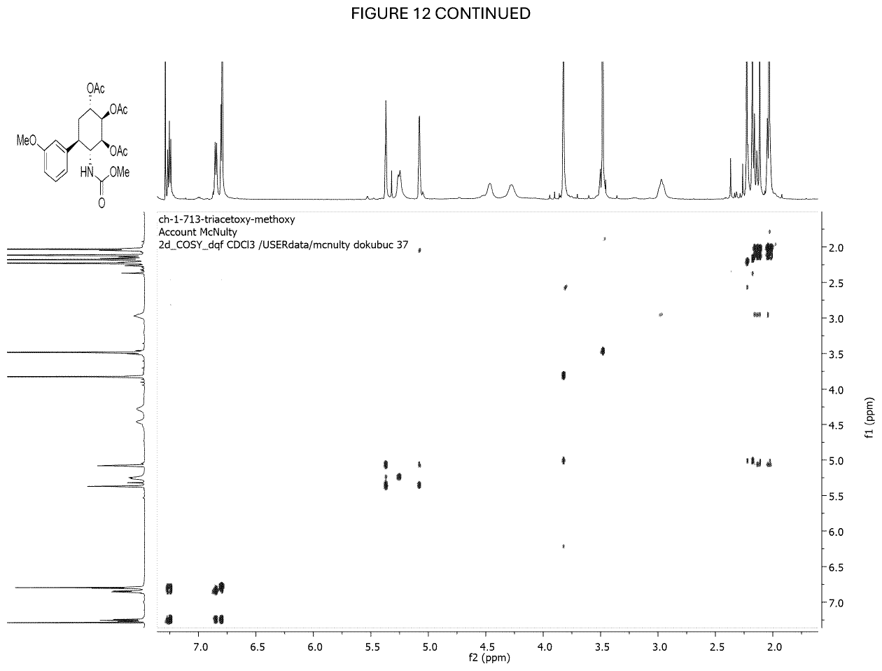

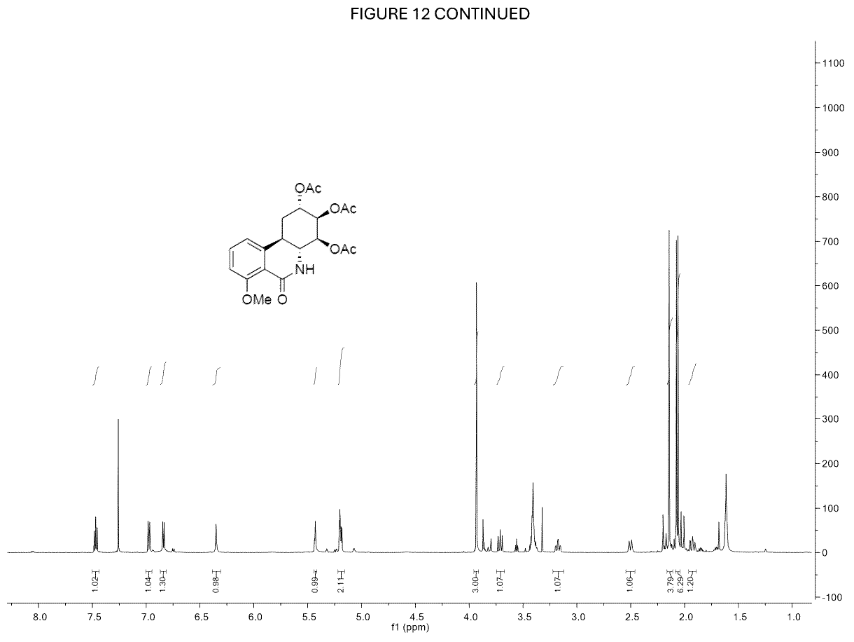

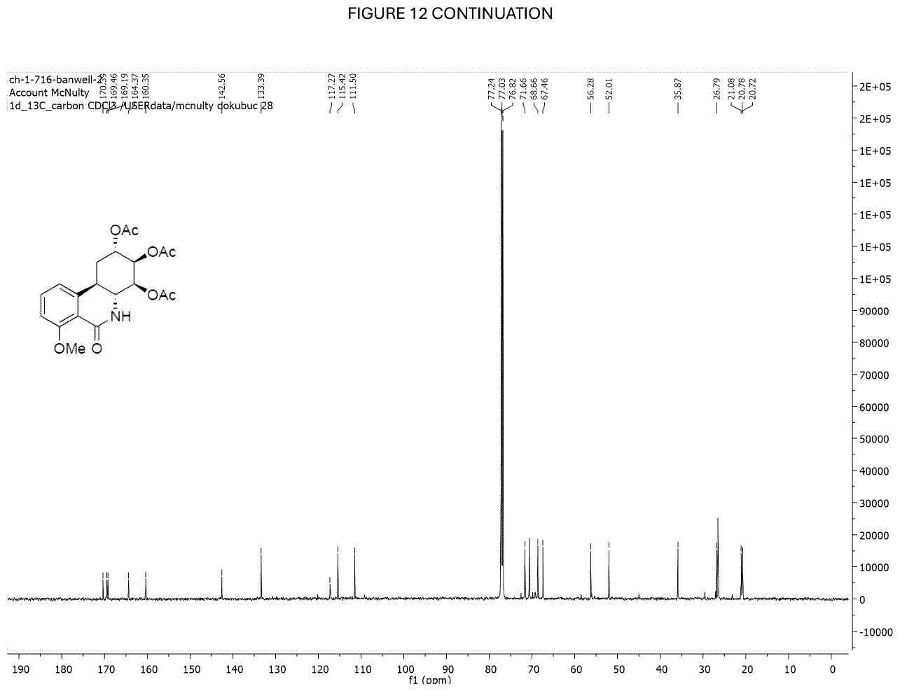

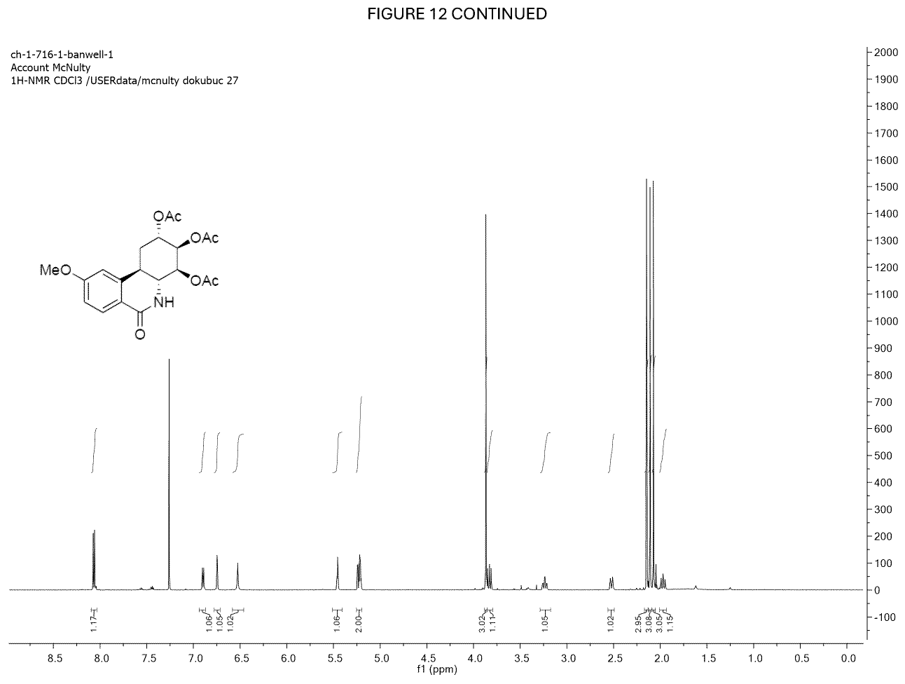

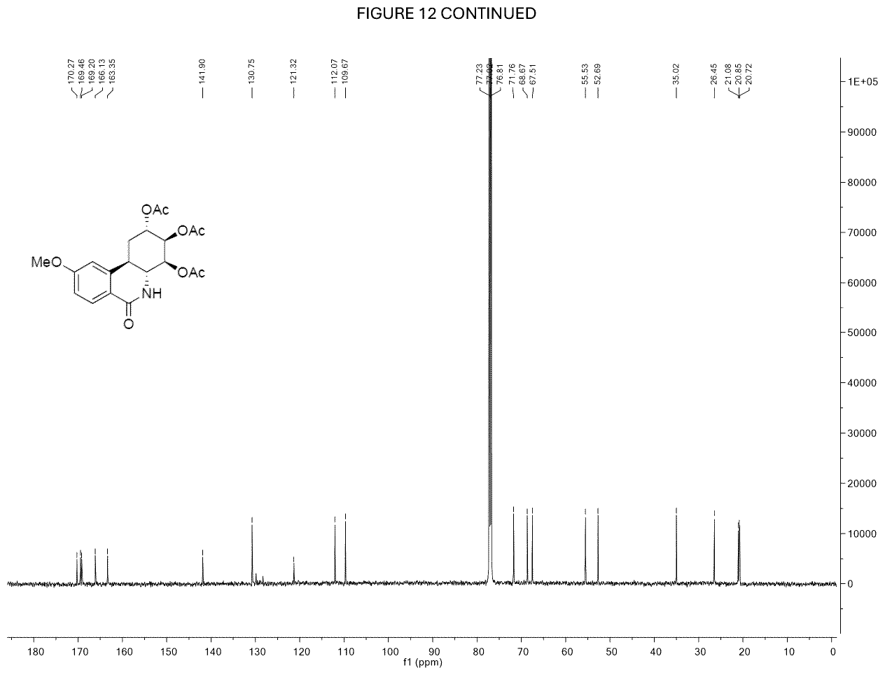

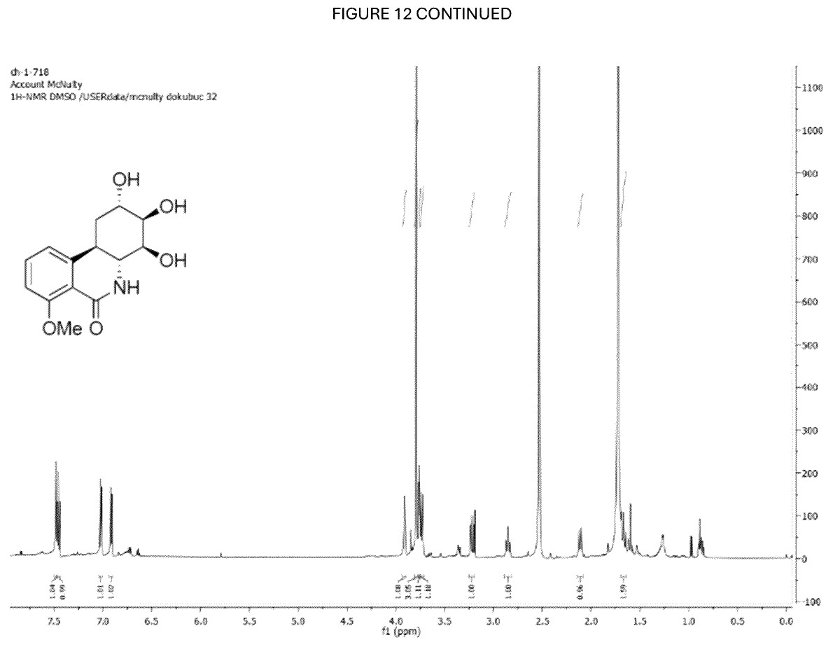

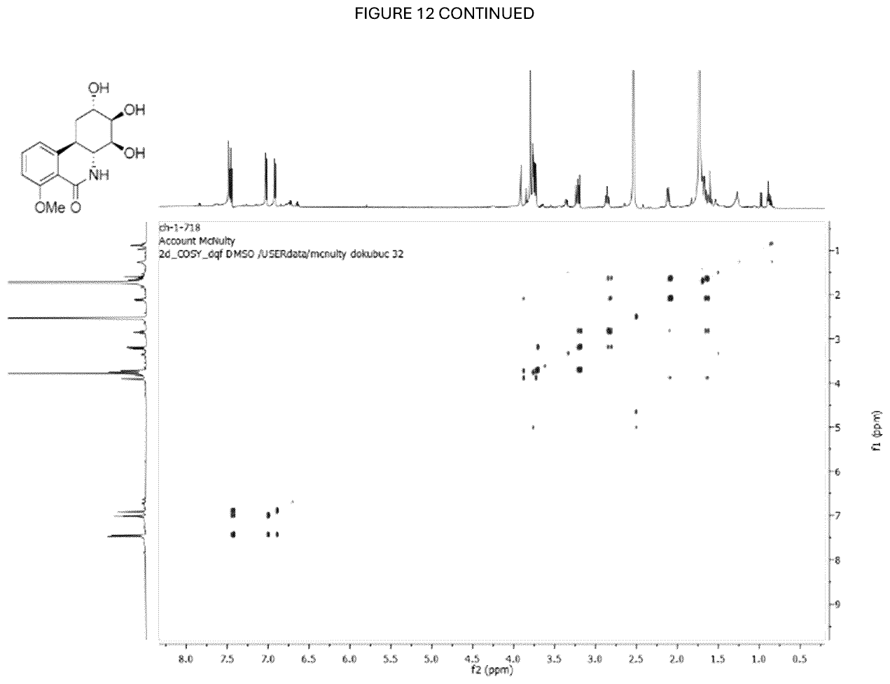

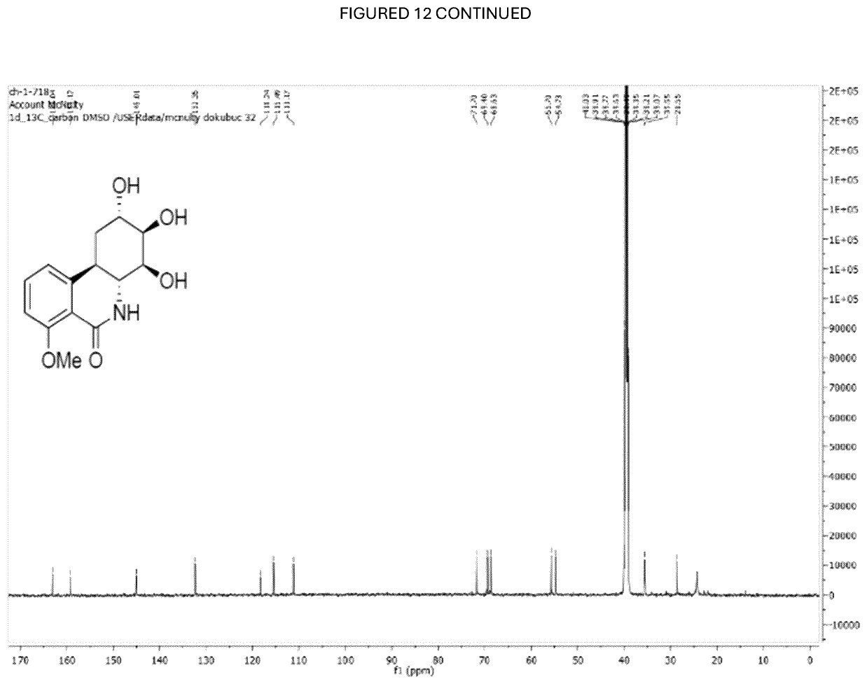

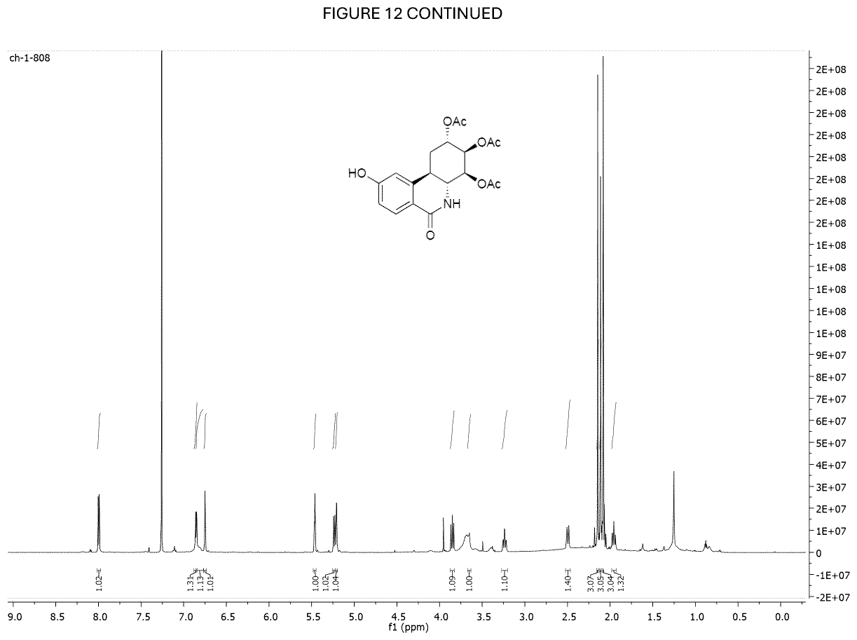

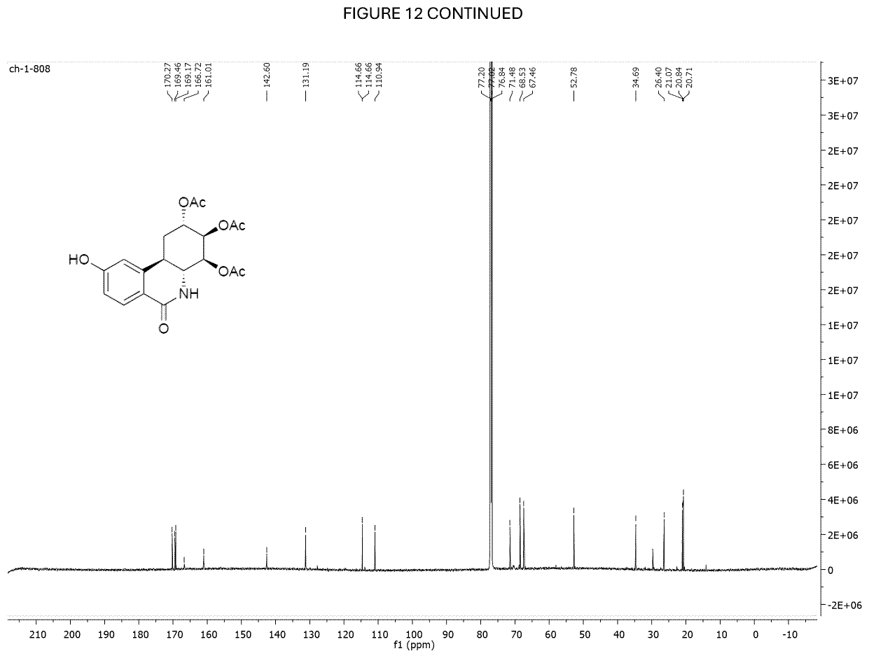

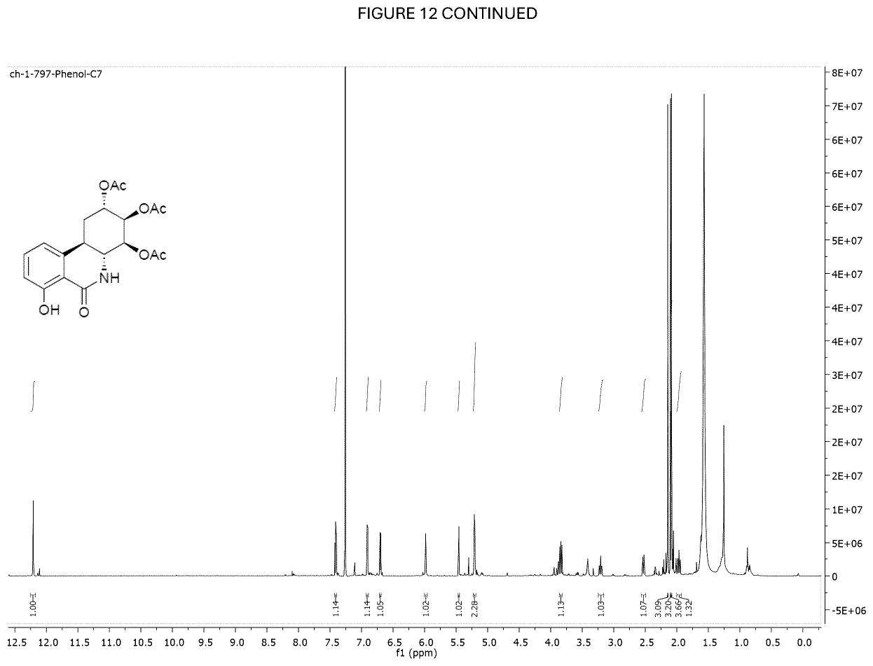

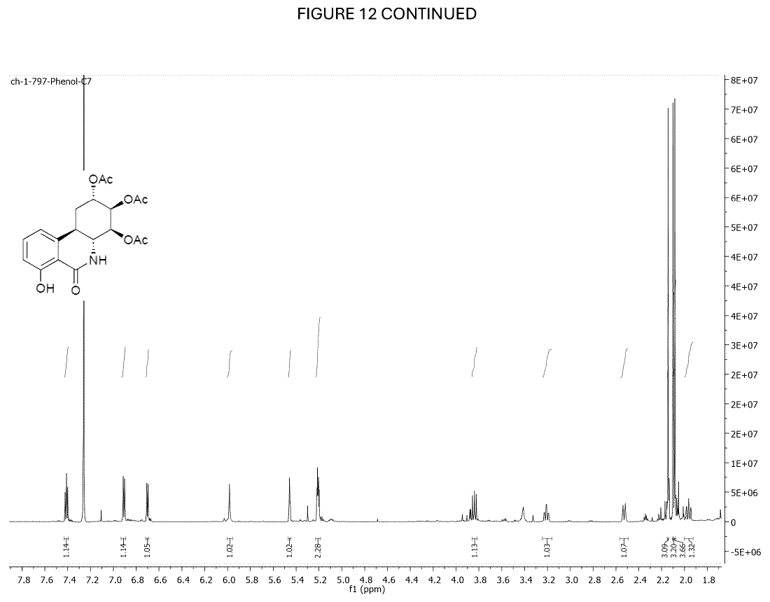

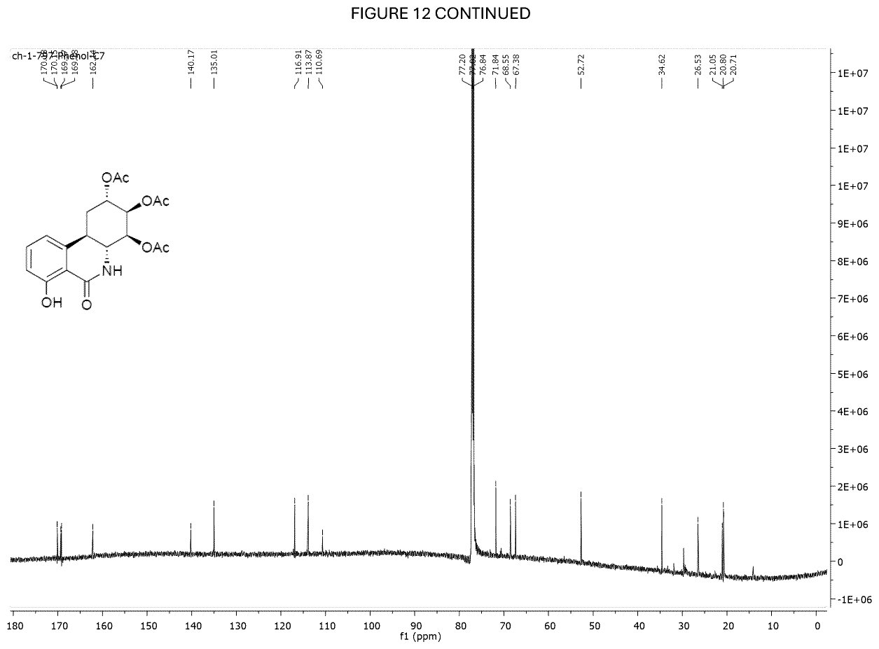

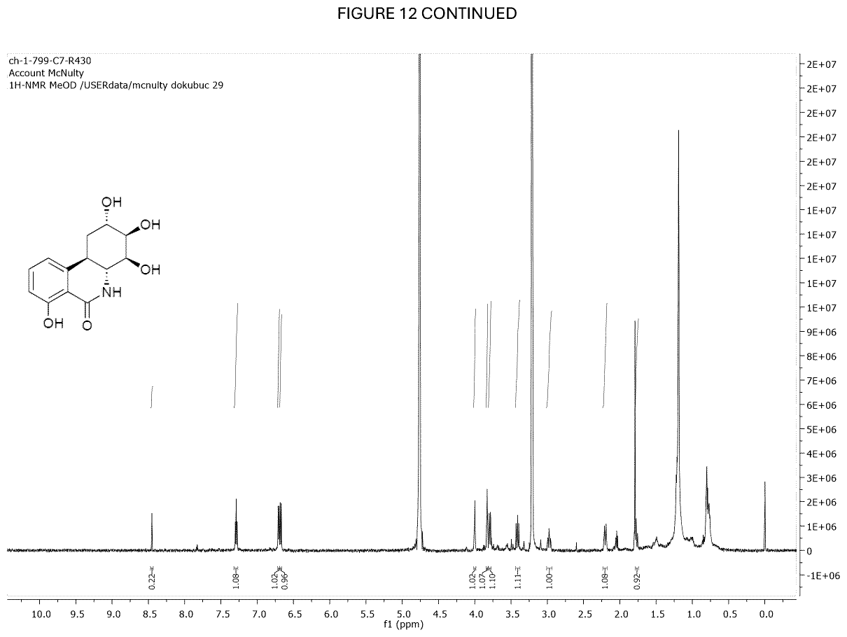

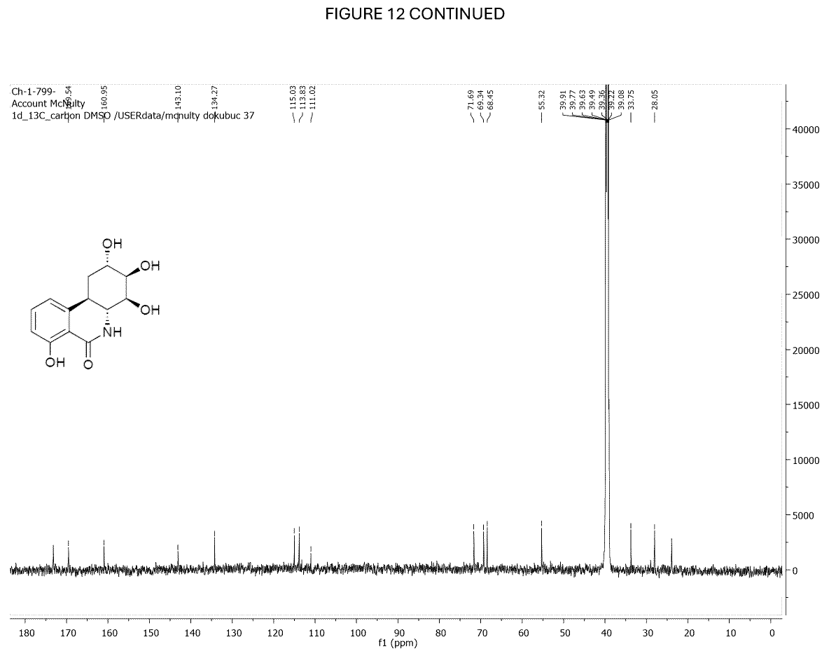

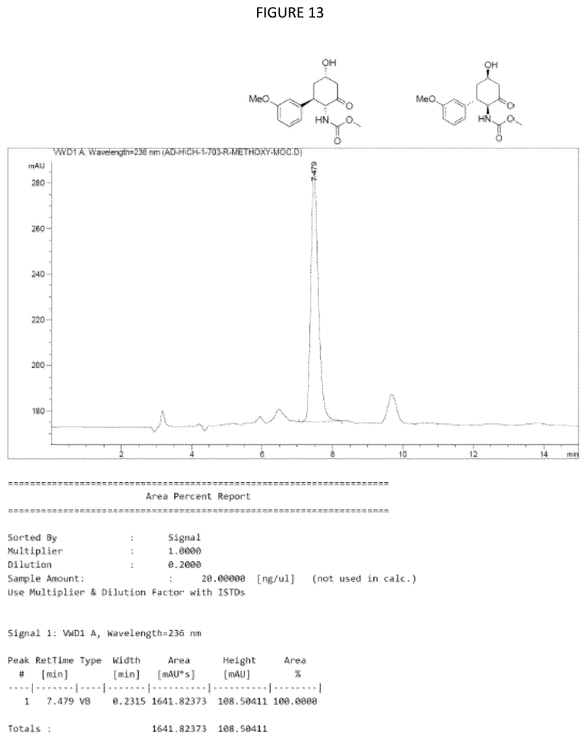

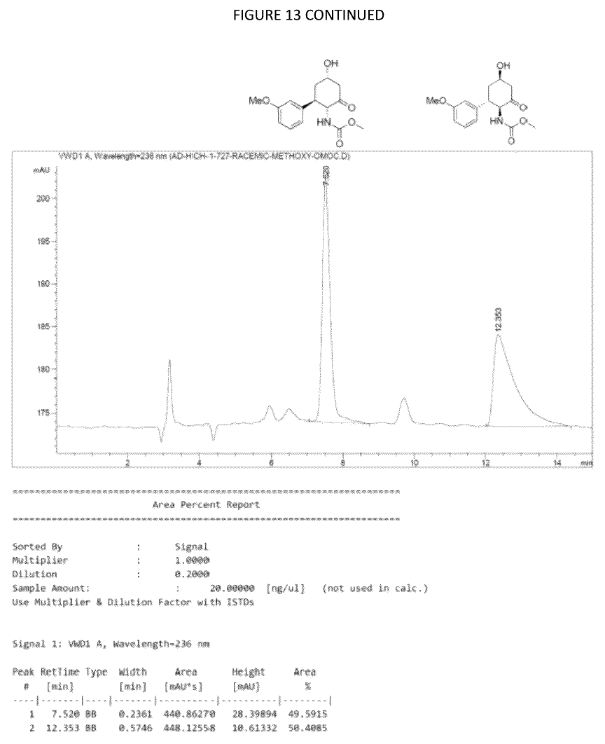

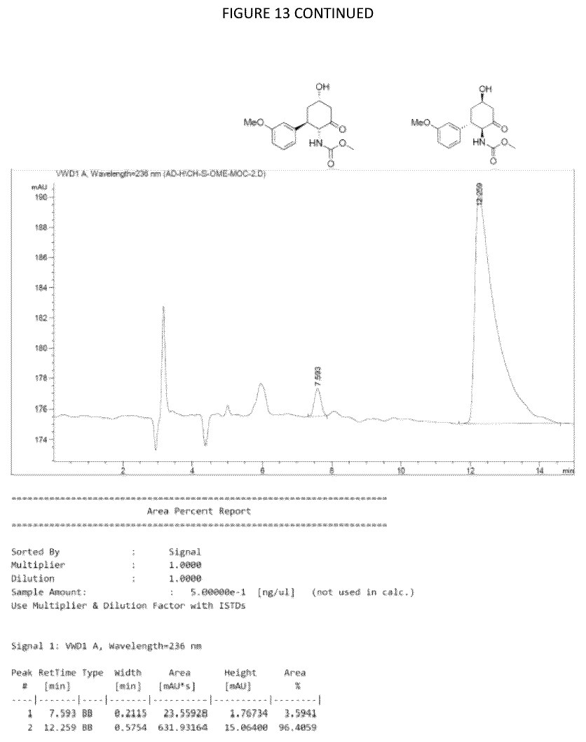

The antiviral efficacy of compound Ia was further investigated in an in vivo mouse model. In order to assess the efficacy of Ia against an acute infection go HSV-1 in vivo, ND4 Swiss mice were infected in the eyes with HSV-1 following light corneal scarification. At the same time that the virus was applied, one group of mice (n=10) were treated with 2 uL of a 50 μM solution of R799 in DMSO and the other group (n=10) were treated with 2 uL DMSO (vehicle), applied to the eyes. The eyes were treated with either vehicle or Ia each day for 4 days. In order to assess the effect of Ia on the course of the infection, the eyes were swabbed daily with a Dacron swab, and the virus eluted in MEM medium. The swab eluate was assessed for the presence of infectious virus by standard plaque assay on rabbit skin cell monolayers. As shown in FIG. 11, the eyes treated with Ia dramatically reduced the yield of infectious virus in the tears compared to vehicle treated eyes. The titer of infectious virus in the vehicle treated eyes peaked at day 2 post-infection) whereas the peak was delayed by one day in the Ia treatment group and was reduced by almost 3-fold.

In conclusion, the development and general antiviral activity of a novel truncated ring-A synthetic alkaloid Ia to HSV-1 and SARSCoV2 is reported. Mechanistic investigations demonstrate activation of a host defense through upregulation of the integrated stress response pathway resulting in autophagy. Importantly, the RNA sequencing profile provides a molecular signature that allows development of a selective antiviral agent, as prolonged and over activation of the ISR can lead to programmed cell death. Modulated activation of the ISR may then lead to decreased protein expression and autophagy, resulting in potent antiviral activity in a selective manner. Upregulation of host-cell defense machinery to viral infection with a therapeutic prototype such as Ia opens the paradigm for the development of a broad-spectrum antiviral agent with little selection toward antiviral drug resistance. The ability to activate innate antiviral immunity through IFN-1 stimulation has been described for small molecule lipids such as bile acids¹⁹ and retinoids,²⁰ involving complex regulatory pathways. This work demonstrates the application of a small molecule antiviral agent to induce innate immunity through activation of the E1F2 kinase (E1F2AK3, also known as PKR-ER or PERK) and upregulation of the ISR, independent of these pathways.

### Methods

### A) Chemical Synthesis and Analysis.

Solvents and reagents: All chemicals and solvents were purchased from Acros, Aldrich, J. T. Baker, Caldeon, Cytec Industries and Fluka and used as received with the following exceptions: deuterated solvents were obtained from ACP Chemicals, Toronto, Canada. Tetrahydrofuran (THF), diethyl ether (Et₂O), and toluene were distilled from sodium/benzophenone under an atmosphere of dry nitrogen; dichloromethane (CH₂Cl₂) was distilled from calcium hydride under an atmosphere of dry nitrogen; methanol (MeOH) was distilled from magnesium turnings under an atmosphere of dry nitrogen; triethylamine (NEt₃), N,N-diisopropylethylamine (Hünig's base) and pyridine were distilled from potassium hydroxide under an atmosphere of dry nitrogen; solid sodium hydride (NaH) was obtained by filtration and washing with n-hexanes.

Reaction handling: All non-aqueous reactions were performed in flame dried round bottom flasks or in non-flame-dried amber 1.5-dram vials. Reactions were magnetically stirred and monitored by thin-layer chromatography (TLC) unless otherwise noted. TLC was performed on Macherey-Nagel silica gel 60 F₂₅₄ TLC aluminum plates and visualized with UV fluorescence quenching and potassium permanganate (KMnO₄) or 2,4-dinitrophenylhydrazine or p-anisaldehyde stains.¹⁷ Concentrations under reduced pressure were performed by rotary evaporation at 40° C. at the appropriate pressure unless otherwise noted. Column chromatographic purification was performed as flash column chromatography with 0.3-0.5 bar pressure using Silicycle silica gel (40-63, 60 Å) or EcoChrom silica gel (12-26, 60 Å). Distilled technical grade solvents were employed. The yields given refer to chromatographically purified and spectroscopically pure compounds unless stated otherwise.

Nuclear Magnetic Resonance (NMR) spectroscopy: ¹H, ¹³C{1H}, DEPTq, COSY, HSQC, and HMBC NMR spectra were obtained on Bruker DRX-500, AV-600, and AV-700 spectrometers. All ¹H NMR spectra were referenced relative to SiMe₄ through a resonance of the employed deuterated solvent or impurity of the solvent; chloroform (7.26 ppm), DMSO (3.33 ppm) and methanol (3.31 ppm) for ¹H NMR; chloroform (77.00 ppm), DMSO (39.52 ppm) and methanol (49.00 ppm) for ¹³C NMR. All NMR spectra were obtained at RT (ca. 22° C.) unless otherwise specified. The data is reported as (s=singlet, d=doublet, t=triplet, m=multiplet or unresolved, br=broad signal, coupling constant(s) in Hz, integration). ¹³C-NMR spectra were recorded with complete ¹H-decoupling. Service measurements were performed by the NMR service team of the Nuclear Magnetic Resonance Facility at McMaster University by Dr. Bob Berno and Dr. Hilary A. Jenkins.

Mass spectrometry: Mass spectrometric analyses were performed as high-resolution ESI measurements on a Waters/Micromass QT of Global Ultima (quadrupole time-of-flight mass spectrometer) or high-resolution EI in a Waters/Micromass GCT (time-of-flight mass spectrometer) instrument by the mass spectrometry service of the McMaster Regional Centre for Mass Spectrometry (MRCMS) at McMaster University by Megan Fair and Leah Allan under the supervision of Dr. Kirk Green.

Enantiomeric ratios: Enantiomeric ratios were determined using an Agilent 1220 Infinity HPLC manual injection with a variable wavelength detector, using a Daicel Chiralpak® AD-H column (150×4.6 mm, 5μ), n-hexane/iPrOH (80:20) as a mobile phase; flow rate 0.75 ml/min, column temperature 25° C., λ236 nm, sample 1 mg/1 ml dissolved in the mobile phase.

Optical rotations: Optical rotations were measured on a Perkin-Elmer 241 MC polarimeter, [α] is given in degcm³g⁻¹dm⁻¹ and c is given in gcm⁻³.

### Synthetic Procedures:

Azidopropan-2-one (11):

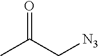

Sodium azide (2.485 g, 38.23 mmol) was added to a solution of 1-bromopropan-2-one (2.54 mL, 31.63 mmol) in acetone (10 mL) and water (20 mL). The reaction mixture was stirred at RT for 24 h and acetone was removed using nitrogen stream. The remaining aqueous solution was extracted with CH₂Cl₂ (2×25 mL). The combined organic extracts were washed with brine, dried over anhydrous Na₂SO₄, and filtered. The filtrate had a nitrogen stream blown over it until all solvent was removed, yielding (2.505 g, 80% yield) the title compound as a colorless liquid. This compound is known and matches the reported spectroscopic data.¹⁷

Tripropyl-(2,2-diethoxyethyl)-Phosphonium Bromide:

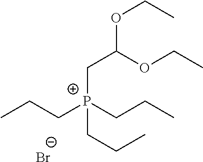 [SMILES: CCC[P+](CCC)(CCC)CC(OCC)OCC ; [Br-]]

A solution of bromoacetaldehyde diethyl acetal (9.60 g, 48.7 mmol) and tri-n-propylphosphine (8.58 g, 53.6 mmol) in THF (27 mL) was heated for 24 h at 60° C. After cooling, the solvent and the residual tri-n-propylphosphine were removed on the rotary evaporator (75° C. at 5 mbar). The obtained yellow oil was dried in vacuo (0.1 mbar) isolating 16.38 g of phosphonium bromide as a white solid. No further purification was necessary. Storage temperature: 0° C. This compound is known and matches the reported spectroscopic data (McNulty & Zepeda-Velázquez, 2014)⁷.

(E)-3-Methoxyphenyl-cinnamaldehyde (10):

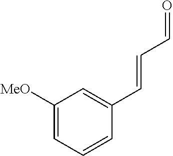 [SMILES: COc1cccc(/C=C/C=O)c1]

3-Methoxybenzaldehyde (1.822 g, 13.40 mmol) and tributyl-(2,2-diethoxyethyl)-phosphonium bromide (5.99 g, 16.7 mmol) were dissolved in anhydrous THF (20 mL). Sodium hydride (0.97 g, 40.3 mmol) was added to the reaction mixture over a period of 10 min, maintaining the temperature below 30° C., and the suspension was stirred for 24 h at RT. Water (25 mL) was added to the mixture, and the mixture was extracted with CH₂Cl₂ (3×25 mL). The extracts were combined and washed again with water (2×25 mL). The organic layers were dried using Na₂SO₄, filtered and concentrated under vacuum to afford diethyl acetal of aldehyde 3.19 g of red oil. 20 mL of 2 M HCl was added and stirred for 1 h at RT. When the reaction was completed, the reaction mixture was extracted with CH₂Cl₂ (3×25 mL). Compound was separated by column chromatography to afford 3-methoxy-cinnamaldehyde 10 (2.063 g, 95% Yield). Data were in accord with reported spectroscopic data,^{18 1}H NMR (600 MHz, CDCl₃) δ 9.67 (d, J=7.7 Hz, 1H, HC═O), 7.41 (d, J=15.9 Hz, 1H, CH═CH—CHO), 7.31 (t, J=7.9 Hz, 1H, Ar—H)), 7.13 (d, J=7.6 Hz, 1H,Ar—H), 7.05 (s, 1H, Ar—H), 6.96 (dd, J=8.2, 1.8 Hz, 1H, CH═CH—CHO 6.67 (dd, J=15.9, 7.7 Hz, 1H, Ar—H), 3.81 (s, 3H, —OCH₃). HRMS (ESI): exact mass calculated for C₁₀H₁₀O₂Na [(M+Na)+], 185.0578; found 185.0566.

(2R,3R,5S)-2-Azido-5-hydroxy-3-(3-methoxyphenyl)cyclohexanone (9):

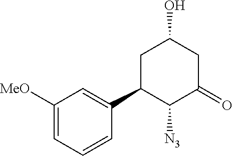 [SMILES: COc1cccc([C@H]2C[C@H](O)CC(=O)[C@@H]2N=[N+]=[N-])c1]

A solution of 3-methoxy-cinnamaldehyde 12 (0.758 g, 4.68 mmol) and (R)-(+)-α,α-diphenyl-2-pyrrolidinemethanol trimethylsilyl ether (0.145 g, 0.45 mmol) in CH₂Cl₂ (6.2 mL) was stirred for 10 min, after which it was cooled to −20° C. Azidopropan-2-one 11 (0.442 g, 4.46 mmol) was added dropwise over 5 min. The brown solution was stirred for 20 min at RT and quinidine (0.145 g, 0.45 mmol) was added in one portion. The reaction was stirred at RT for 24 h, after which TLC (CH₂Cl₂/MeOH 98:2) showed full conversion. The CH₂Cl₂ was carefully evaporated (30° C., 32 mbar) yielding a brown oil that was purified by flash chromatography (eluent CH₂Cl₂/MeOH 100:0 to 95:5,) to afford product 9 (0.671 g, 55% yield). [α]²³_{D}=+87 (c=0.82, MeOH, 1=1 dm). ¹H NMR (600 MHz, CDCl₃) δ 7.30 (t, J=7.9 Hz, 1H, Ar—H,), 6.89 (d, J=7.6 Hz, 1H, Ar—H), 6.86-6.84 (m, 1H, Ar—H), 6.83 (s, 1H, Ar—H), 4.58 (m, 1H, CH—OH), 4.10 (d, J=12.0 Hz, 1H, CH—N₃), 3.82 (s, 3H,—OCH₃), 3.46 (td, J=12.1, 4.3 Hz, 1H, Ph—CH), 2.83-2.69 (m, 2H, CH₂CO), 2.27-2.13 (m, 2H, Ph—CH—CH₂), 1.72 (s, 1H,—OH). ¹³C NMR (151 MHz, CDCl₃) δ 202.92, 159.99, 142.09, 130.02, 119.52, 113.59, 112.61, 71.21, 67.88, 55.27, 48.14, 44.70, 39.33. HRMS (ESI): exact mass calculated for C₁₃H₁₅N₃O₃Na [(M+Na)⁺], 284.1011; found 284.1006

(2S,3S,5R)-2-Azido-5-hydroxy-3-(3-methoxyphenyl)cyclohexanone (ent-9):

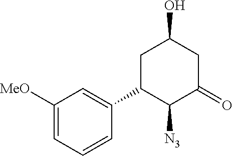

The title compound was synthesized following the above procedure but employing(S)-(+)-α,α-diphenyl-2-pyrrolidinemethanol trimethylsilyl ether as catalyst. [α]²³_{D}=−82 (c=1, MeOH, 1=1 dm).

Methyl ((1R,2R,4S)-4-hydroxy-2-(3-methoxyphenyl)-6-oxocyclohexyl)carbamate (13):

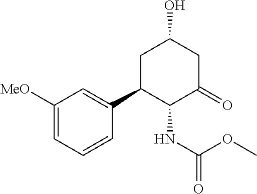 [SMILES: COC(=O)N[C@H]1C(=O)C[C@@H](O)C[C@@H]1c1cccc(OC)c1]

In a 50 ml RBF, azide 9 (0.391 g, 1.5 mmol), dimethyl dicarbonate (0.600 g, 4.5 mmol) and 10% Pd/C (0.12 g, 0.10 mmol) were suspended in methanol (15 mL). The vessel was sealed and subjected to hydrogen balloon (1 atm) with vigorous stirring for 12 h, after which TLC (CH₂Cl₂/MeOH 95:5) showed full conversion. The suspension was filtered through a celite® pad and carefully evaporated (20° C., 0.1 mbar). The translucent grey oil was purified by flash chromatography (eluent CH₂Cl₂/MeOH 100:0 to 97:3) giving the product as a translucent oil carbamate 13 (0.325 g, 75% yield) [α]²³_{D}=−23 (c=2, MeOH, 1=1 dm). e.e.: τmajor=7.49 min, τminor=12.3 min (>99.5% e.e.), AD-H column (150×4.6 mm, 5μ), n-hexane/iPrOH (80:20) as a mobile phase; flow rate 0.75 ml/min, column temperature 25° C., λ236 nm, sample 1 mg/1 ml dissolved in the mobile phase. ¹H NMR (600 MHz, CDCl₃) δ 7.24 (t, J=7.9 Hz, 1H, Ar—H), 6.86 (d, J=7.5 Hz, 1H, Ar—H), 6.80 (d, J=12.7 Hz, 1H, Ar—H), 6.78 (dd, J=8.2, 2.3 Hz, 1H, Ar—H), 5.25 (d, J=8.7 Hz, 1H), 4.61 (t, J=9.2 Hz, 1H), 4.53 (m, 1H), 3.78 (s, 3H, Ar—OCH₃), 3.48 (s, 3H, NHCOOCH₃), 3.28 (dd, J=19.7, 8.5 Hz, 1H, Ph—CH), 3.04-2.95 (bs, 1H, OH), 2.83 (d, J=13.1 Hz, 1H, COCH₂), 2.70 (d, J=13.9 Hz, 1H, COCH₂), 2.19 (t, J=11.2 Hz, 2H, Ph—CH—CH₂). ¹³C NMR (151 MHz, CDCl₃) δ 206.01, 159.64, 157.02, 142.30, 129.62, 119.76, 113.56, 112.33, 68.17, 63.17, 55.16, 52.16, 48.19, 45.44, 40.29. HRMS (ESI): exact mass calculated for C₁₅H₁₉NO₅Na [(M+Na)⁺], 316.1161; found 316.1158

(2S,4R,4aR,10aR)-4,9-Bis(3-methoxyphenyl)-1,2,3,4,4a,5,10,10a-octahydrophenazin-2-ol (18):

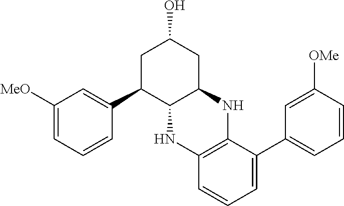 [SMILES: COc1cccc(-c2cccc3c2N[C@@H]2C[C@@H](O)C[C@H](c4cccc(OC)c4)[C@H]2N3)c1]

By-product 18 (0.062 g, 10% yield). [α]²³_{D}=+97 (c=1, MeOH, 1=1 dm). ¹H NMR (600 MHz, CDCl₃) δ 7.40 (t, J=7.9 Hz, 1H, Ar—H), 7.31 (t, J=7.8 Hz, 1H, Ar—H), 7.03 (d, J=7.5 Hz, 1H, Ar—H), 7.00 (s, 1H, Ar—H), 6.95 (d, J=8.3 Hz, 1H, Ar—H), 6.90 (d, J=7.5 Hz, 1H, Ar—H), 6.86 (d, J=8.9 Hz, 1H, Ar—H), 6.84 (s, 1H, Ar—H,), 6.75 (t, J=7.7 Hz, 1H, Ar—H), 6.61 (d, J=7.5 Hz, 1H, Ar—H), 6.40 (d, J=7.9 Hz, 1H, Ar—H), 4.49-4.29 (bs, 1H, NH) 4.15-4.11 (m, 1H, Ph—CH), 3.41 (td, J=3.2, 12.0 Hz, 1H, Ph—CH—CH(NH)—CH—NH), 2.21 (d, J=14.0 Hz, 1H, Ph—CH—CH₂), 2.13 (d, J=15.0 Hz, 1H, CH(NH)—CH₂), 1.96-1.87 (m, 2H, CH(NH)—CH₂, Ph—CH—CH₂), 1.70 (s, 1H, CH—OH). ¹³C NMR (151 MHz, CDCl₃) δ 159.96 (2C), 144.20, 140.16, 132.64, 130.04, 129.80, 129.20, 127.83, 121.33, 120.60, 120.37, 119.57, 114.03, 113.93, 113.91(2C), 113.65, 112.34, 67.03, 56.14, 55.28 (2C), 49.52, 39.43, 39.26, 36.68. HRMS (ESI): exact mass calculated for C₂₆H₂₈NO₃Na [(M+Na)⁺], 439.1988; found 439.1978. Note: When 0.15 equiv of 10 mol % Pd/C was used and reaction was stirred to 32 h, by-product 18 was not observed.

Methyl ((1S,2S,4R)-4-hydroxy-2-(3-methoxyphenyl)-6-oxocyclohexyl) carbamate (ent-13):

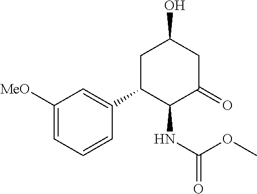 [SMILES: COC(=O)N[C@@H]1C(=O)C[C@H](O)C[C@H]1c1cccc(OC)c1]

The title compound was made from ent-9. [α]²³_{D}=+21 (c=2, MeOH, 1=1 dm). Enantiomeric ratio was determined using chiral HPLC: minor=7.59 min, major=12.3 min (>99.5% e.e.), AD-H column (150×4.6 mm, 5μ), n-hexane/iPrOH (80:20) as a mobile phase; flow rate 0.75 ml/min, column temperature 25° C., λ236 nm, sample 1 mg/1 ml dissolved in the mobile phase.

Methyl ((1R,2R,3R)-3-hydroxy-3′-methoxy-1,2,3,6-tetrahydro-[1,1′-biphenyl]-2-yl)carbamate (14)

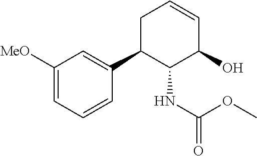 [SMILES: COC(=O)N[C@H]1[C@H](O)C=CC[C@@H]1c1cccc(OC)c1]

In a 50 mL RBF, the carbamate 13 (0.360 g, 1.23 mmol) was dissolved in CH₂Cl₂ (18 mL) under nitrogen and cooled in an ice bath. Methanesulfonyl chloride (0.123 mL, 1.59 mmol) was added in one portion, and then Hünig's base (0.642 mL, 3.69 mmol) was added dropwise. The resulting solution was stirred for 10 h at RT, then poured into water (10 mL) and the layers separated. The organic phase was washed with 1 M HCl (1 mL) then brine (10 mL) and dried over Na₂SO₄. Concentration under reduced pressure gave the crude product as a translucent oil (0.305 g, 95% yield). This product was used with out further purification. In a 50 mL RBF, ketone (0.305 g, 1.10 mmol) was dissolved in THF 15 ml, and cooled to 0° C. and added Lithium tri-tert-butoxyaluminum hydride (0.846 g, 3.30 mmol). After 12 h of stirring, saturated aqueous NH₄Cl was added to quench the reaction. The product was extracted with EtOAc (3×15 mL) and the combined organic layer was washed with brine (10 mL) and dried over Na₂SO₄. The solvent was removed under reduced pressure and the residue was purified by column chromatography (CH₂Cl₂/MeOH; 100:0 to 97:3) to afford the product 14 (0.291 g, 95% yield) as a translucent oil. [α]²³_{D}=−104.3 (c=1, MeOH, 1=1 dm). ¹H NMR (600 MHz, CDCl₃) δ 7.24 (t, J=6.9 Hz, 1H, Ar—H), 6.79 (m, 2H, Ar—H), 6. 6.76 (dt, J=3.9, 1.8 Hz, 1H, Ar—H), 5.82-5.75 (m, 1H, CH2—CH═), 5.72 (dt, J=3.7, 2.1 Hz, 1H, ═CH—CH—OH), 4.59 (brs, 1H, NH), 4.33 (br s, 1H, CH—OH), 4.25 (brs, —OH) 3.85 (ddd, 1H,, J=13.4, 7.8, 6.5 Hz, 1H, CH—NH), 3.80(s, 3H, Ar—OCH₃), 3.55 (s, 3H, HNCOO—CH₃), 2.86 (td, J=10.3, 6.5 Hz, 1H, Ar—CH), 2.42-2.29 (m, 2H, Ar—CH—CH₂). ¹³C NMR (151 MHz, CDCl₃) δ 160.03, 158.54, 142.65, 130.05, 129.40, 126.92, 120.00, 113.51, 112.57, 74.09, 58.60, 55.22, 52.44, 44.82, 34.96. HRMS (ESI): exact mass calculated for C₁₅H₁₉NO₄Na [(M+Na)⁺], 300.1212; found 300.1208

(1S,2R,3S,4R,5R)-4-((Methoxycarbonyl)amino)-5-(3-methoxyphenyl)cyclohexane-1,2,3-triyl triacetate (15):

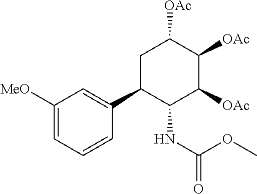 [SMILES: COC(=O)N[C@H]1[C@H](OC(C)=O)[C@H](OC(C)=O)[C@@H](OC(C)=O)C[C@@H]1c1cccc(OC)c1]

In a 50 mL RBF, vinyl alcohol 14 (0.260 g, 0.94 mmol) was dissolved in CH₂Cl₂ (20 mL) along with NaHCO₃ (0.158 g, 1.88 mmol). To the stirred suspension was added m-CPBA (0.422 g, 1.88 mmol) at RT. The resulting suspension was stirred vigorously for 24 h. A 20% (w/v) aqueous solution of sodium sulfite (10 mL) was added, and the resulting two-phase mixture was stirred vigorously for 15 min. The two layers were separated, and the aqueous layer was extracted with CH₂Cl₂ (2×10 mL). The combined organic layers were washed with a 20% (w/v) aqueous solution of sodium sulfite (10 mL) and a 5% (w/v) aqueous solution of NaHCO₃ (2×10 mL), dried with anhydrous Na₂SO₄ and evaporated under reduced pressure (20° C., 32 mbar) to give a 4:1 diastereomeric mixture (0.234 g) of compounds by TLC and which was used for next step without further purification.

To a solution of epoxide mixtures (0.234 g, 0.80 mmol) in 5.1 mL of water was added sodium benzoate (0.009 g, 0.06 mmol). The mixture was heated at 90-95° C. for 16 h. TLC showed full conversion. The solution was cooled to RT, the water was removed in vacuo, and the light pink residue which single trihydroxy compound (0.249 g) was used for next step, without further purification.

To a solution of trihydroxy compound (0.140 g, 0.80 mmol) in pyridine (0.386 mL, 4.80 mmol) was added acetic anhydride (0.243 mL, 4.80 mmol). The reaction mixture was stirred at RT for 16 h. After that the pyridine was removed in vacuo (0.1 mbar). The residue was dissolved in EtOAc (20 mL) and washed with saturated NaHCO₃ (2×10 mL) and water (10 mL). The solvent was removed in vacuo and the product purified by flash column chromatography (CH₂Cl₂/MeOH; 100:0 to 98:2) to afford triacetate 15 (0.316 g, over 3 steps 77% yield). [α]²³_{D}=−12 (c=1, MeOH, 1=1 dm).

¹H NMR (600 MHz, CDCl₃) δ 7.22 (t, J=8.0 Hz, 1H, Ar—H), 6.84 (t, J=8.0 Hz, 1H, Ar—H), 6.80-6.75 (m, 2H, Ar—H), 5.37 (t, J=2.9 Hz, 1H, CH—OAc), 5.23 (d, J=9.2 Hz, 1H, CH—OAc), 5.15-4.96 (m, 1H, CH—OAc), 4.44 (d, J=33.5 Hz, 1H, NH), 4.25 (m, 1H, CHNH), 3.80 (s, 3H Ar—OCH₃), 3.46 (s, 3H, O═COCH₃), 2.95 (m, 1H. Ar—CH), 2.38 (s, 3H, O═C—CH₃), 2.17 (s, 3H, O═C—CH₃), 2.17-2.12 (m, 1H,), 2.03-2.02(m, 1H, Ar—CH—CH₂), 2.01 (s, 3H, O═C—CH₃). HRMS (ESI): exact mass calculated for C₂₁H₂₇NO₉Na [(M+Na)⁺], 460.1584; found 460.1578.

(2S,3R,4S,4aR,10bR)-9-Methoxy-6-oxo-1,2,3,4,4a,5,6,10b-octahydrophenanthridine-2,3,4-triyl triacetate (16):

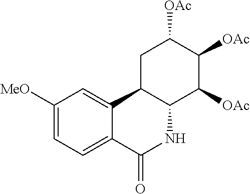 [SMILES: COc1ccc2c(c1)[C@H]1C[C@H](OC(C)=O)[C@@H](OC(C)=O)[C@@H](OC(C)=O)[C@@H]1NC2=O]

Into a 50 mL RBF, compound 15 (0.140 g, 0.32 mmol) and DMAP (0.117 g, 0.96 mmol) were dissolved in CH₂Cl₂ (8 mL) at 0° C. A 1.0 M solution of Tf₂O in CH₂Cl₂ (1.597 mL, 1.60 mmol) was added dropwise to the reaction mixture over a period of 10 min. The reaction was stirred for 16 h at RT. The solvent was evaporated, and the residue treated with a mixture of THF (5 mL) and 1 M HCl (1 mL). After stirring for 1 h at RT, the mixture was partitioned between a saturated aqueous solution of NaHCO₃ (1 mL) and CH₂Cl₂ (20 mL). The organic phases were combined, dried with anhydrous Na₂SO₄ and concentrated. The Regio isomers (compounds 16 and 17) were purified by flash column chromatography (CH₂Cl₂/MeOH; 100:0 to 98:2). Compound 16 (major), 0.066 g, 51% yield; Rf=0.35 (Hexane/ethyl acetate, 1:1); [α]²³_{D}=+34 (c=0.1, MeOH, 1=1 dm).¹H NMR (600 MHz, CDCl₃) δ 8.096 (d, J=8.65 Hz, 1H), 6.939 (dd, J=8.54, 2.09 HZ, 1H), 6.778 (m, 1H), 6.582 (s, 1H), 5.513 (t, J=2.94 HZ, 1H), 5.254 (m, 2H), 3.884 (s, 3H), 3.855 (dd, J=11.1, 12.4 HZ, 1H), 3.287 (d, J=3.74, 12.23 HZ, 1H), 2.561 (dt, J=3.3, 14.6 HZ, 1 H), 2.70 (s, 3H), 2.126 (s, 3H), 2.088 (s, 3H), 2.008 (m, 1H); ¹³C NMR (151 MHz, CDCl₃) δ 170.27, 169.46, 169.20, 166.13, 163.35, 141.90, 130.75, 121.32, 112.07, 109.67, 77.23, 77.02, 76.81, 71.76, 68.67, 67.51, 55.53, 52.69, 35.02, 26.45, 21.08, 20.85, 20.72. HRMS (ESI): exact mass calculated for C₂₀H₂₃NO₈Na [(M+Na)+], 428.1321; found 428.1316.

(2S,3R,4S,4aR,10bR)-7-Methoxy-6-oxo-1,2,3,4,4a,5,6,10b-octahydrophenanthridine-2,3,4-triyl triacetate (17):

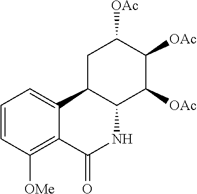 [SMILES: COc1cccc2c1C(=O)N[C@H]1[C@H](OC(C)=O)[C@H](OC(C)=O)[C@@H](OC(C)=O)C[C@H]21]

Compound 17 (minor), 0.019 g, 15% yield, Rf=0.30 (Hexane/ethyl acetate 1:1), [α]²³_{D}=+41 (c=1, MeOH, 1=1 dm). ¹H NMR (600 MHz, CDCl₃) δ 7.50 (m, 1H), 7.00(d, J=8.57 HZ, 1H), 6.869 (d, J=7.76 HZ, 1H), 6.386 (s, 1H), 5.441 (t, 3H), 5.219 (m, 2H), 3.957 (s, 3H), 3.746 (t, J=11.35 HZ, 1H), 3.223 (td, J=3.98, 12.6 HZ, 1H), 2.545 (dt, J=3.4, 14.3 HZ, 1H), 2.163 (s, 3H), 2.08 (s, 3H), 2.06 (s, 3H), 1.962 (m, 1H). ¹³C NMR (151 MHz, CDCl₃) δ 170.39, 169.46, 169.19, 164.37, 160.35, 142.56, 133.39, 117.27, 115.42, 111.50, 71.66, 68.66, 67.46, 56.28, 52.01, 35.87, 26.79, 21.08, 20.78, 20.72. HRMS (ESI): exact mass calculated for C₂₀H₂₃NO₈Na [(M+Na)⁺], 428.1321; found 428.1314.

(2S,3R,4S,4aR,10bR)-2,3,4-Trihydroxy-9-methoxy-1,3,4,4a,5,10b-hexahydrophenanthridin-6(2H)-one (5):

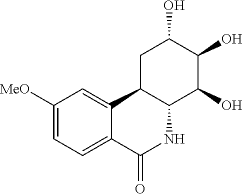 [SMILES: COc1ccc2c(c1)[C@H]1C[C@H](O)[C@@H](O)[C@@H](O)[C@@H]1NC2=O]

Compound 16 (0.005 g (0.012 mmol) and potassium carbonate (0.002 g, 0.01 mmol) were dissolved in MeOH (1 mL) and stirred at RT. TLC analysis showed full conversion after 6 h. The mixture was concentrated under a flow of N₂. The white product was dissolved in 9:1 CH₂Cl₂/MeOH and filtered through a pad of silica to afford 9-metoxy trihydroxy compound 5 (0.0032 g, 96% yield). [α]²³_{D}=+52 (c=0.25, MeOH, 1=1 dm). 1H NMR (700 MHz, DMSO) δ 7.81 (d, J=8.5 Hz, 1H), 6.90 (d, J=8.5, Hz, 1H), 6.87 (s, 1H), 6.82 (s, 1H), 5.14 (bm, 3H), 3.91 (d, J=2.4 Hz, 1H), 3.82 (s, 3H), 3.76-3.71 (m, 2H), 2.95 (dd, J=11.9, 9.3 Hz, 1H), 2.18 (dt, J=13.0, 3.2 Hz, 1H), 1.70 (td, J=13.0, 2.2 Hz, 1H). ¹³C NMR (176 MHz, DMSO) δ 164.60, 162.35, 144.41, 129.45, 122.07, 111.69, 109.23, 71.72, 69.89, 68.66, 55.37, 55.07, 40.06, 34.53, 28.03. HRMS (ESI): exact mass calculated for C₁₄H₁₇NO₅Na [(M+Na)⁺], 302.1004; found 302.0986.

(2S,3R,4S,4aR,10bR)-2,3,4-Trihydroxy-7-methoxy-1,3,4,4a,5,10b-hexahydrophenanthridin-6(2H)-one (7):

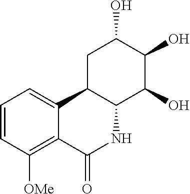 [SMILES: COc1cccc2c1C(=O)N[C@H]1[C@H](O)[C@H](O)[C@@H](O)C[C@H]21]

Compound 17 (0.005 g (0.012 mmol) and potassium carbonate (0.002 g, 0.01 mmol) were dissolved in MeOH (1 mL) and stirred at RT. TLC analysis showed full conversion after 6 h. The mixture was concentrated under a flow of N₂. The white product was dissolved in 9:1 CH₂Cl₂/MeOH and filtered through a pad of silica 7-methoxy trihydroxy compound 7 (0.0032 g, 96% yield). [α]²³_{D}=+56 (c=0.25, DMSO, 1=1 dm). ¹H NMR (600 MHz, DMSO) δ0 7.47 (s, 1H, CONH), 7.45 (t, J=8.0 Hz, 2H, Ar—H), 7.02 (d, J=8.5 Hz, 1H, Ar—H) 6.91 (d, J=7.8 Hz, 1H, Ar—H), 3.91 (dd, J=3.2, 6,25 Hz, 1H, CH₂CH—OH), 3.79 (s, 3H, Ar—OMe), 3.77-3.75 (m, 1H, CH2CHOHCHOH), 3.25-3.19 (dd, J=10.2, 3.0 Hz, 1H, CHNHCHOH) 2.85 (td, J=12.3, 3.8 Hz, 1H,), 2.11 (dd, J=10.0, 3.2 Hz, 1H), 1.67 (td J=3.2 13.7, 1H). ¹³C NMR (151 MHz, DMSO) δ 163.04, 159.12, 145.01, 132.35, 118.24, 115.49, 111.17, 71.70, 69.40, 68.63, 55.70, 54.73, 35.55, 28.55. HRMS (ESI): exact mass calculated for C₁₄H₁₇NO₅Na [(M+Na)⁺], 302.1004; found 302.0988.

(2S,3R,4S,4aR,10bR)-9-Hydroxy-6-oxo-1,2,3,4,4a,5,6,10b-octahydrophenanthridine-2,3,4-triyl triacetate (19):

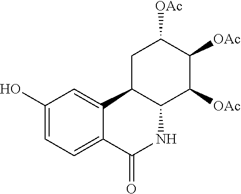 [SMILES: CC(=O)O[C@H]1[C@@H]2NC(=O)c3ccc(O)cc3[C@H]2C[C@H](OC(C)=O)[C@H]1OC(C)=O]

Compound 16 (0.050 g, 0.123 mmol) was dissolved in 1:1 ratio of dry CH₂Cl₂ and dry Benzene. After addition of Aluminium chloride (0.049 g, 0.369 mmol) under argon at 0° C. and n-Bu4N⁺I⁻ (0.2 7 g, 0.738 mmol) was added under argon was added to the reaction mixture over a period of 10 min at 0° C. The reaction mixture turned red and was stirred for 3 hours at RT. After confirming full conversion of starting material with TLC, the reaction mixture was quenched with water, and stirred for 2 N HCl for 0.5 h, and was extracted with EtOAc (3× 10 mL). The combined organic phase was washed with brine, dried over Na₂SO₄, filtered through Dowex® 50WX8 hydrogen form and concentrated in vacuo and purified by flash column chromatography (CH₂Cl₂/MeOH; 100:0 to 98:2) to afford phenolic compound 19 (0.042 g, 88% Yield). [α]²³_{D}=+45 (c=0.9, MeOH, 1=1 dm). ¹H NMR (700 MHz, CDCl₃) δ 7.99 (t, J=7.8 Hz, 1H, ArH), 6.86 (dd, J=8.4, 1.9 Hz, 1H, ArH), 6.85-6.77 (bs, 1H, CONH), 6.75 (s, 1H, ArH), 5.46 (t, J=3.1 Hz, 1H, CH—OAc), 5.23 (dd, J=10.8, 2.9 Hz, 1H, CH—OAc), 5.21 (dd, J=5.9, 2.8 Hz, 1H, CH—OAc), 3.85 (dd, J=12.5, 11.2 Hz, 1H CH—N), 3.67 (dd, J=25.2, 9.1 Hz, 1H, Ph—OH), 3.24 (td, J=12.7, 3.5 Hz, 1H, Ar—CH), 2.50 (dt, J=14.4, 3.0 Hz, 1H, CH₂), 2.15 (s, 3H, COCH₃), 2.11 (s, 3H, COCH₃), 2.08 (m, 3H, COCH₃) 1.98-1.93 (m, 1H, CH₂). ¹³C NMR (176 MHz, CDCl₃) δ 170.27, 169.46, 169.17, 166.72, 161.01, 142.60, 131.19, 114.66, 114.66, 110.94, 71.48, 68.53, 67.46, 52.78, 34.69, 26.40, 21.07, 20.84, 20.71. HRMS (ESI): exact mass calculated for C₁₉H₂₁NO₈Na [(M+Na)⁺], 414.1165; found 414.1156.

(2S,3R,4S,4aR,10bR)-2,3,4,9-Tetrahydroxy-1,3,4,4a,5,10b-hexahydrophenanthridin-6(2H)-one (6):

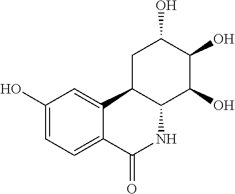 [SMILES: O=C1N[C@H]2[C@H](O)[C@H](O)[C@@H](O)C[C@@H]2c2cc(O)ccc21]

Compound 19 (0.0047 g (0.012 mmol) and potassium carbonate (0.002 g, 0.010 mmol) in MeOH (1 mL) was stirred at RT until a white solid precipitated. TLC analysis showed full conversion after 6 h. The mixture was concentrated under a flow of N₂. The white product was dissolved in 9:1 CH₂Cl₂/MeOH and filtered through a pad of silica. The white solid was recrystallized from methanol to afford trihydroxy compound 6 (0.003 g, 94% yield). [α]²³_{D}=+31 (c=0.25, MeOH, 1=1 dm). ¹H NMR (700 MHz, DMSO) δ 7.75 (d, J=8.54 Hz, 1H), 6.75(bs, 1H), 6.74 (dd, J=8.54, 2.3 Hz, 1H), 6.71 (s, 1H), 3.94 (dd, J=3.1, 5.6 Hz, 1H), 3.77-3.74 (m, 2H), 3.36 (dd, J=12.4, 9.4 Hz, 1H), 2.94 (td, J=12.4, 3.5 Hz, 1H), 2.13-2.08 (dt, J=3.5, 13.2 Hz, 1H), 1.70 (td, J=13.2, 2.5 Hz, 1H). C NMR (176 MHz, DMSO) δ 165.28, 161.45, 144.84, 130.03, 120.93, 113.74, 110.61, 72.14, 70.37, 69.10, 55.42, 34.78, 28.55. HRMS (ESI): exact mass calculated for C₁₃H₁₅NO₅Na [(M+Na)⁺], 288.0848; found 288.0842.

(2S,3R,4S,4aR,10bR)-7-Hydroxy-6-oxo-1,2,3,4,4a,5,6,10b-octahydrophenanthridine-2,3,4-triyl triacetate (20):

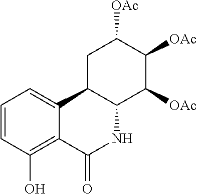 [SMILES: CC(=O)O[C@H]1[C@@H]2NC(=O)c3c(O)cccc3[C@H]2C[C@H](OC(C)=O)[C@H]1OC(C)=O]

Compound 17 (0.005 g, 0.012 mmol) was dissolved in 1:1 ratio of dry CH₂Cl₂ 0.5 mL and dry Benzene 0. 5 mL. After addition of Aluminium chloride (0.005 g, 0.037 mmol) under argon at 0° C. and n-Bu₄N⁺I⁻ (0. 0.027 g, 0.074 mmol) was added under argon slowly to the reaction mixture over a period of 10 min. The reaction mixture turned red and was stirred for 1 h at RT. After confirming full conversion with TLC, reaction mixture was quenched with water, and stirred for 2 N HCl for 0.5 h, and was extracted with EtOAc (3×10 mL). The combined organic phase was washed with brine, dried over Na₂SO₄, filtered through Dowex® 50WX8 hydrogen form and concentrated in vacuo and purified by flash column chromatography (CH₂Cl₂/MeOH; 100:0 to 98:2) to afford compound 20 (0.0043 g, 89% Yield). [α]²³_{D}=+45 (c=0.5, MeOH, 1=1 dm). ¹H NMR (700 MHz, CDCl₃) δ 12.21 (s, 1H Ph—OH), 7.41 (t, J=7.75 Hz, 1H, Ar—H), 6.91 (d, J=8.4 Hz, 1H), 6.70 (d, J=7.6 Hz, 1H, Ar—H), 5.98 (s, 1H, CON—H), 5.47-5.44 (m, 1H, CH—OAc), 5.23-5.19 (m, 2H, CH—OAc, CH—OAc), 3.84 (dd, J=12.7, 10.9 Hz, 1H, CH—N), 3.21 (td, J=12.7, 3.7 Hz, 1H, Ph—CH), 2.53 (d, J=14.5 Hz, 1H, CH₂), 2.15-2.14 (m, 3H, COCH₃), 2.10 (d, J=5.3 Hz, 3H, COCH₃), 2.08 (m, 3H, COCH₃), 1.97 (ddd, J=15.0, 9.5, 3.1 Hz, 1H, CH₂). ¹³C NMR (176 MHz, CDCl₃) δ 170.18, 170.15, 169.37, 169.18, 162.14, 140.17, 135.01, 116.91, 113.87, 110.69, 71.84, 68.55, 67.38, 52.72, 34.62, 26.53, 21.05, 20.80, 20.71. HRMS (ESI): exact mass calculated for C₁₉H₂₁NO₈Na [(M+Na)⁺], 414.1165; found 414.11564.

(2S,3R,4S,4aR,10bR)-2,3,4,7-Tetrahydroxy-1,3,4,4a,5,10b-hexahydrophenanthridin-6(2H)-one (Ia):

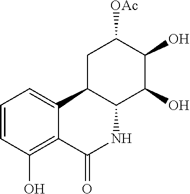 [SMILES: CC(=O)O[C@H]1C[C@@H]2c3cccc(O)c3C(=O)N[C@H]2[C@H](O)[C@@H]1O]

Compound 20 (0.0043 g (0.011 mmol) and potassium carbonate (0.002 g, 0.010 mmol) were MeOH (1 mL) and stirred at RT until a white solid precipitated. TLC analysis showed full conversion after 6 h. The mixture was concentrated under a flow of N₂. The white product was dissolved in 9:1 CH₂Cl₂/MeOH and filtered through a pad of silica. The white solid was recrystallized from methanol to afford trihydroxy compound Ia (0.0026 g, 96% yield). [α]²³_{D}=+28 (c=0.25, MeOH, 1=1 dm). ¹H NMR (600 MHz, CD₃OD) δ 8.45 (s, 1H, Ph—OH, 80% was deuterated), 7.29 (t, J=7.81 Hz, 1H, ArH), 6.70 (d, J=7.7 Hz, 1H, ArH), 6.68 (d, J=8.3 Hz, 1H, ArH), 4.00 (dd, J=2.9, 6.3 Hz, 1H, CH₂CHOH), 3.84-3.82 (m, 1H CH₂ CHCHOH), 3.79 (dd, J=10.3, 2.8 Hz, 1H, CHOH), 3.44-3.38 (dd, J=10.3, 12.7 Hz, 1H, CHNH), 2.98 (td, J=11.0, 3.69 Hz, 1H, PhCH), 2.20 (dt, J=13.4, 3.19 Hz, 1H, CH₂), 1.77 (m, 1H, CH₂). ¹³C NMR (151 MHz, DMSO) δ 169.54, 160.95, 143.10, 134.27, 115.03, 113.83, 111.02, 71.69, 69.34, 68.45, 55.32, 33.75, 28.05. HRMS (ESI): exact mass calculated for C₁₃H₁₅NO₅Na [(M+Na)⁺], 288.0848; found 288.0840.

### B) Antiviral Activity Assessment.

### Generation of Neural Progenitor Cells (NPCs).

HiPSCs were cultured in mTeSR1-plus medium supplemented with dual SMAD inhibitors SB 431542 and LDN 193189 to promote neural induction. After 8-10 days, neural rosettes were manually isolated, transferred into Matrigel coated plates and cultured in StemDiff Neural Progenitor Medium (STEMCELL Technologies) for the expansion of NPCs.

HiPSCs were cultured in mTeSR1-plus medium supplemented with dual SMAD inhibitors SB431542 (10 μM) and LDN193189 (100 nM) (NPS/Dual-SMAD) to induce neuroectoderm formation. After 8-10 days, neural rosettes were manually isolated, transferred into Matrigel coated plates and cultured in StemDiff Neural Progenitor Medium (STEMCELL Technologies) for the expansion of NPCs.

### Generation of Organoids From Brain Organoids.

Human iPSC (hiPSC) lines 01SD and 9001 were employed to generate brain-like organoids. Organoids were generated as previously described (Abrahamson EE et al., Modeling Aβ42 Accumulation in Response to Herpes Simplex Virus 1 Infection: 2D or 3D? J Virol. 2021 Mar. 1;95(5)) with some modification in the initial part regarding the generation of spheroids containing neural rosettes. hiPSCs cultured with mTeSR™ plus medium (STEMCELL Technologies) in Matrigel-coated 6-well plates were detached with Accutase and then dissociated into single cell suspension by gently pipetting. They were seeded into low attachment U-bottom 96-well plates at the density of 9000 cells/well, in mTeSR plus medium supplemented with Rho-associated protein kinase inhibitor (ROCK inhibitor) Y27632 (STEMCELL™) to generate embryoid bodies (EBs). After three days, to induce neuroectoderm, the medium was switched to Essential 6 medium (ThermoFisher Scientific) supplemented with dual SMAD inhibitors SB431542 10 μM (MilliporeSigma S4317-5MG) and LDN193189 100 nM (MilliporeSigma SML055-9-25 MG. Cultures were observed on daily basis.

On day 8, differentiating EBs were rinsed with Dulbecco's Modified Eagle Medium: Nutrient Mixture F-12 (DMEM/F12, Gibco 11330-032) and cultured in neuronal medium (DMEM/F12 supplemented with 1× MEM Nonessential Amino Acid supplement (MEM-NEAA, CORNING® 25-025-CI), 1× Glutamax (Gibco 35050-061), 1× N2 supplement (Gibco 17502-048) and 1 μg/ml Heparin (STEMCELL™ 07980)) in untreated 10-cm petri dishes. These plates were placed on an orbital shaker in the incubator at 70 rpm (Orbi-Blotter™). Culture medium was halved every three days.

On day 20, the culture medium was replaced with cortical organoid differentiation medium I ((CODMI: DMEM:F12/Neurobasal (1:1 v/v) supplemented with 1× Glutamax, 1× B-27 (VitA [−]), 0.5× Non-essential amino acids, 0.5× N-2, Insulin (2.5 μg) and 1× penicillin-streptomycin (P/S). On day 25, CODM-I medium was replaced with cortical organoid differentiation medium II (CODM-II: DMEM/F12-Neurobasal (1:1 v/v) supplemented with 1× glutamax, 1× B-27 (VitA [+]), 0.5× Non-essential amino acids, 0.5× N-2, Insulin (2.5 μg), BDNF (10 ng/ml), and 1× P/S. Culture medium was changed every 3 days. On day 42, CODM-II medium was replaced with BrainPhys™ Neuronal medium (StemCell Technologies).

### Infection of Neuronal Progenitor Cells (NPCs).

Monolayer cultures of NPCs were infected at an MOI of 0.1 with an HSV-1 recombinant virus expressing the reporter genes EGFP and RFP under the control of the HSV-1 promoters ICP0 and gC, respectively (Ref). Two hours after the infection the inocula were removed, cells were washed and cultured in StemDiff Neural progenitor Medium supplemented with the above described R430-analogs at the concentration of 10 μM or ACV at 50 μM. Cells were analyzed by flow cytometry at day 3 p.i.

### Infection of Brain Organoids.

Fourteen-week old brain organoids were infected singularly in U-bottom low attachment 96-well plates with an HSV-1 DualF construct (6000 pfu/organoid). After 2 hours, the inocula were removed, the organoids were washed and cultured in BrainPhys medium supplemented with the tested compounds (10 μM) or ACV (50 μM). The organoids that were infected in the presence of ACV were pretreated with the antiviral for 24 hours.

### Organoids Viability.

Organoids viability was analyzing using calcein AM assay (BioLegend; catalogue #425201), according to manufacturer's instructions. Briefly, organoids were exposed singularly in Eppendorf tube containing Calcein-AM 0.01 μM and incubated at 37° C. After 20 min, organoids were transferred in prewarmed culture medium and incubated for additional 10 min. After incubation, fluorescence signals were recorded using a LEICA DMIL LED Fluorescent microscope through a 0.16NA 4x air objective.

The corrected total cell fluorescence (CTCF) was obtained by measuring EGFP fluorescence in 799-treated and untreated organoids, and normalizing for whole organoid area using equation CTCF=integrated density−(whole organoid area x mean fluorescence of background readings).

### RNA Sequencing.

NPCs were cultured in StemDiff Neural progenitor Medium, containing R430, 799, (10 μM) or vehicle. All assays were conducted in triplicate. Cells were harvested after 72 hours and the cellular RNA was extracted (RNeasy Mini Kits, Qiagen) and quantified using Agilent 4200 TapeStation (Agilent Technologies). Total RNA libraries were generated using the Illumina TruSeq Stranded Total RNA Sample Preparation Guide, Revision E. The first step involved the removal of ribosomal and mitochondrial RNA using biotinylated, target-specific oligos combined with Ribo-Zero rRNA removal beads. Following purification, remaining RNA was fragmented using divalent cations under elevated temperature, which were then copied into first strand cDNA using reverse transcriptase and random primers, followed by second strand cDNA synthesis using DNA Polymerase I and RNase H. Subsequently, a single adenosine base was added to each of the cDNA fragments, followed by ligation of an adapter. The products were purified and enriched with PCR to create the final cDNA library. The cDNA libraries were validated using KAPA Biosystems primer premix kit with Illumina-compatible DNA primers and Qubit 2.0 fluorimeter. Quality was examined using an Agilent Bioanalyzer Tapestation 2200. The cDNA libraries were pooled at a final concentration 1.8 pM. Cluster generation and 100 bp paired-read dual-indexed sequencing was performed on Illumina NextSeq 500 (Children's Hospital of Pittsburgh, University of Pittsburgh).

Sequencing read quality was assessed using fastQC v0.11.4 and CLCbio v11.0.1 software. The average number of reads per sample was 39.5 million (SD=4.8 million reads). Sequences were trimmed based on quality score using the modified-Mott trimming algorithm as implemented in CLC bio software, using a trim cutoff error probability of 0.05. Ambiguous bases were trimmed using a post trim maximal ambiguous base cutoff of 2. The trimmed reads were then mapped to the human genome GRCh38/hg38, using sequence and annotation provided by Ensembl (release 82). Approximately 92% of reads were mapped in pairs (SD=1.14) across all samples, and 97.7% of reads were mapped in total (SD=0.45). The data were deposited in NCBI's Gene Expression Omnibus database (GSE201156).

Quantitative RT-PCR (qRT-PCR).

Quantitative reverse transcription PCR (qRT-PCR) was used to determine gene expression analysis using Taqman Assays (Thermo Fisher, US) specific for TP53 (ID Hs01034249_m1), SIRT1 (Hs01009006_m1) and BCL2 (Hs00608023_m1) genes and ATF4 with primers set: FWD 5′TCAAACCTCATGGGTTCTCC3′ (SEQ ID NO: 1) and REV: 5′GTGTCATCCAACGTGGTCAG3′ (SEQ ID NO: 2). Every sample had 3 or 4 biological replicates. Reverse transcription reaction was obtained from 100 ng of total RNA using random hexamers and SuperScript IV (Thermo Fisher). The conditions for the RT reaction were priming at 65° C. for 5 min, following 55° C. for 10 min and 80° C. for 10 min; then held on 4° C. 2 μL of diluted cDNA (1:3) was added to Luna Universal Probe qPCR Master Mix or Luna Universal qPCR Master Mix (NBE, USA) to the total volume of 10 uL. PCR was performed in CFX96 (BioRad, USA) under following conditions: 95° C. for 60 s, followed by 40 cycles of 95° C. for 15 s and 30° C. for 1 min. The levels of gene expression were determined using Ct (threshold cycle). The ΔCt was calculated by subtracting the Ct of GAPDH (FWD: 5′ACCCACTCCTCCACCTTTG3′ (SEQ ID NO: 3), REV: 5′CTCTTGTGCTCTTGCTGG3′ (SEQ ID NO: 4) from the Ct of interest gene. ΔΔC was calculated by subtracting ΔC of the reference sample from the ΔC of the control samples. Fold change was presented by the following equation 2 ^{ΔΔ}Ct.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### FULL CITATIONS FOR DOCUMENTS REFERRED TO IN THE APPLICATION

1. (a) Dittmar, M.; Lee, J. S.; Whig, K.; Segrist, E.; Li, M. Brinda, K.; Castellana, L.; Ayyanathan, K.; Cardenas-Diaz, F. L.; Morrisey, E. E.; Truitt, R.; Yang, W.; Jurado, K.; Samby, K.; Ramage, H.; Schultz, D. C.; Cherry, S. Cell Reports 2021, 35, 108959 (b) Cragg, G. M.; Grothaus, P. G.; Newman, D. J. Chem. Rev. 2009, 109, 3012-3043. (c) Sayed, A. M.; Khattab, A. R.; AboulMagd, A. M.; Hassan, H. M.; Rateb, M. E.; Zaid, H.; Abdelmohsen, U. R. RSC Adv. 2020, 10, 19790-19802. (d) Cully, M. Nat. Rev. Drug Disc. 2022, 21, 1-5.
2. (a) Jin, Z.; Yao, G. Nat. Prod. Rep. 2019, 36, 1462-1488, and prior reports in this series. (b) Evidente, A.; Kireev, A.; Jenkins, A.; Romero, A.; Steelant, W.; Van Slambrouck, S.; Kornienko, A. Planta Med. 2009, 75, 501-507. (c) Pettit, G. R.; Gaddamidi, V.; Cragg, G. M.; Herald, D. L.; Sagawa, Y. J. Chem. Soc. Chem. Commun. 1984, 1693-1694. (d) Pettit, G. R.; Gaddamidi, V.; Cragg, G. M. J. Nat. Prod. 1984, 47, 1018-1020. (e) Pettit, G. R.; Gaddamidi, V.; Herald, D. L.; Singh, S. B.; Cragg, G. M.; Schmidt, J. M.; Boettner, F. E.; Williams, M; Sagawa, Y. J. Nat. Prod. 1986, 49, 995-1002. (f) Pettit, G. R.; Pettit, G. R. III; Backhaus, R. A.; Boyd, M. R.; Meerow; A. W. J. Nat. Prod. 1993, 56, 1682-1687. (g) McLachlan, A.; Kekre, N.; McNulty, J.; Pandey, S. Apoptosis 2005, 10, 619-630. (h) Kekre, N.; Griffin, C.; McNulty, J.; Pandey, S. Cancer Chemother. Pharmacol. 2005, 10, 619-630. (i) Pandey, S.; Kekre, N.; Naderi, J., McNulty, J. Artifi. Cells, Blood. Sub. & Biotech. 2005, 33, 279-295. (j) Siedlakowski, P.; McLachlan-Burgess, A.; Griffin, C.; Tirumalai, S.; McNulty, J.; Pandey, S. Canc. Biol. & Therap. 2008, 7, 376-384. (k) Ingrassia, L.; Lefranc, F.; Dewelle, J.; Pottier, L.; Mathieu, V.; Spiegl-Kreinecker, S.; Sauvage, S.; El Yazidi, M.; Dehoux, M.; Berger, W.; van Quaquebeke, E.; Kiss, R. J. Med. Chem. 2009, 52, 1100-1114. (l) Lefranc, F.; Sauvage, S.; Van Goietsnoven, G.; Megalizzi, V.; Lamoral-Theys, D.; Debeir, O.; Spiegl-Kreinecker, S.; Berger, W.; Mathieu, V.; Decaestecker, C.; Kiss, R. Mol. Cancer Therap. 2009, 8, 1739-1750.
3. (a) Gabrielsen, B.; Monath, T. P.; Huggins, J. W.; Kefauver, D. F.; Pettit, G. R.; Groszek, G.; Hollingshead, M.; Kirsi, J. J.; Shannon, W. M.; Schubert, E. M.; DaRe, J.; Ugarkar, B.; Ussery, M. A.; Phelan, M. J J. Nat. Prod. 1992, 55, 1569-1581. (b) Renard-Nozaki, J.; Kim, T.; Imakura, Y.; Kihara, M.; Kobayashi, S. Res. Virol. 1989, 140, 115-28. (c) L. D'Aiuto, K. Williamson, P. Dimitrion, J. McNulty, C. E. Brown, C. B. Dokuburra, A. J. Nielsen, W. J. Lin, P. Piazza, M. E. Schurdak, J. Wood, R. H. Yolken, P. R. Kinchington, D. C. Bloom and V. L. Nimgaoknar, Antiviral Res., 2017, 142, 136; (d) L. D'Aiuto, J. McNulty, C. Hartline, M. Demers, R. Kalkeri, J. Wood, L. McClain, A. Chattopadhay, Y. Zhi, J. Naciri, A. Smith,. Yolken, K. Chowdari, C. Zepeda-Vela\acute{ }quez, C. B. Dokuburra R, E. Marques, R. Ptak, P. Kinchington, S. Watkins, M. Prichard, D. Bloom and V. L. Nimgaonkar, Sci. Rep., 2018, 8, 16662. (e) Li, S.-Y.; Chen, C.; Zhang, H.-Q.; Guo, H.-Y.; Wang, H.; Wang, L.; Zhang, X.; Hua, S.-N.; Yu, J.; Xiao, P.-G.; Li, R.-S.; Tan, X. Antiviral Res. 2005, 67, 18-23. (f) Tan, S.; Banwell, M. G.; Ye, W.-C.; Lan, P.; White, L. V. Chem Asian J. 2022, 17, e202101215
4. (a) J. McNulty, C. Zepeda-Velazquez, Angew. Chem. Int. Ed. 2014, 53, 8450-8454. (b) Revu, O.; Zepeda-Velázquez, C.; Nielsen, A. J.; McNulty, J.; Yolken, R. H.; Jones-Brando, L. Chem Select 2016, 1, 5895-5899.
5. (a) McNulty, J.; D'Aiuto, L.; Zhi, Y.; McClain, L.; Zepeda Velázquez, C.; Ler, S.; Jenkins, H. A.; Yee, M. B.; Piazza, P.; Yolken, R. H.; Kinchington, P. R.; Nimgaonkar, V. L. ACS Med. Chem. Lett. 2016, 7, 46-50. (b) C. E. Brown, T. Kong, J. F. Britten, N. H. Werstiuk, J. McNulty, L. D'Aiuto, M. Demers and V. L. Nimgaonkar, ACS Omega, 2018, 3, 11469.
6. Nieto-Garcia, O.; Alonso, R. Org. Biomol. Chem. 2013, 11, 515-522.
7. Banwell, M. G.; Bisset, B. D.; Busato, S.; Cowden, C. J.; Hockless, D. C. R.; Holman, J. W.; Read, R. W. Chem. Commun. 1995, 2551-2553.
8. Kreipl, A. T.; Reid, C.; Steglich, W. Org. Lett., 2002, 4, 3287-3288.
9. Wang, K.; Mahalingam, G.; Hoover, S. E,; Mont, E. K.; Holland, S. M.; Cohen, J. I.; Straus, S. E. J. Virol. 2007, 81, 6817-6826.
10. Honess, R. W.; Watson, D. H. J. Virol. 1977, 21, 584-600.
11. Zheng, W.; Klammer, A. M.; Naciri, J. N.; Yeung, J.; Demers, M.; Milosevic, J.; Kinchington, P. R.; Bloom, D. C,; Nimgaonkar, V. L.; D'Aiuto, L. J. Virol. 2020, 94, e0094-20.
12. Kapałczyńska, M.; Kolenda, T.; Przybyła, W.; Zajączkowska, M.; Teresiak, A.; Filas, V.; Ibbs, M.; Bliźniak, R.; Łuczewski, Ł.; Lamperska, K. Arch Med Sci. 2018,14, 910-919.
13. Costa-Mattiolo, M.; Walter, P. Science, 2020, 368, eaat5314.
14. Pakos-Zebrucka, K.; Koryga, I.; Mnich, K.; Ljujic, M.; Samali, A.; Gorman, A. M. EMBO Rep. 2016, 17, 1374-1395.
15. Humeau, J.; Leduc, M.; Cerrato, G.; Loos, F.; Kepp, O.; Kroemer, G. Cell Death Dis. 2020, 11, 433.
16. Ahmad, L.; Mostowy, S.; Sancho-Shimizu, V. Front. Cell Dev. Biol. 2018, 6, 00155.
17. McNulty, J., Zepeda-Velazquez, C., Angew. Chem. Int. Ed. 2014, 53, 8450-8454. Wang, L., Hubert, J. A., Lee, S. J., Pan, J., Qian, S., Reitman, M. L., Strack, A. M., Weingarth, D. T., MacNeil, D. J., Weber, A. E., Edmondson S. D., Bioorganic & Medicinal Chemistry Letters, 2011, 21, 2911-2915.

1. A compound of Formula (I): 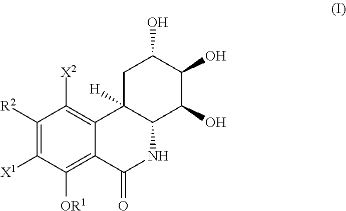
or a pharmaceutically acceptable salt, solvate and/or prodrug thereof;
wherein
R¹ is selected from H, Me and Bn;
R² is selected from H, OH, OMe, OBn, CF₃ and OCF₃;
X¹ is selected from H, F, Cl, Br and I; and
X² is selected from H, F, Cl, Br and I.

2. The compound of claim 1, wherein R¹, R², X¹ and X² are H.

3. A compound selected from 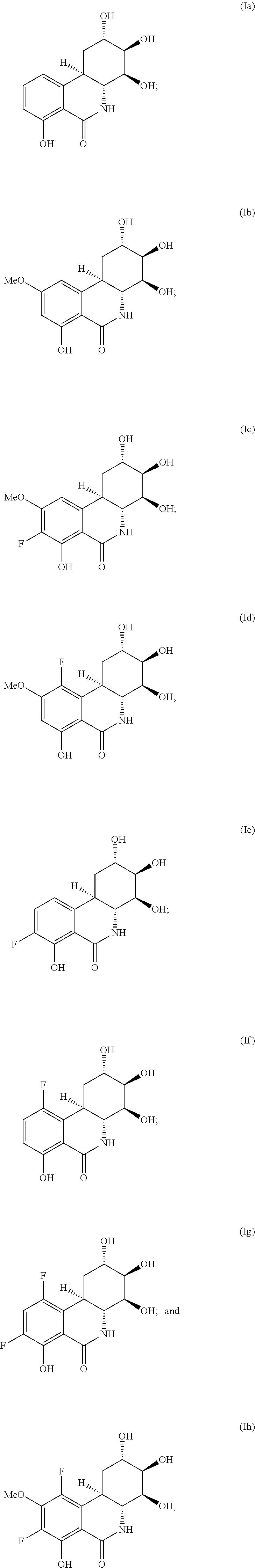
or a pharmaceutically acceptable salt, solvate and/or prodrug thereof.

4. A pharmaceutical composition comprising one or more compounds of claim 1 or a pharmaceutically acceptable salt, solvate and/or prodrug thereof, and one or more pharmaceutically acceptable carriers.

5. A pharmaceutical composition comprising one or more compounds of claim 3 or a pharmaceutically acceptable salt, solvate and/or prodrug thereof, and one or more pharmaceutically acceptable carriers.

6. A method of treating a disease, disorder or condition treatable by activating the integrated stress response via upregulation of eukaryotic initiation factor 2α (eIF2α), the method comprising administering a therapeutically effective amount of one or more compounds of claim 1 or a pharmaceutically acceptable salt, solvate and/or prodrug thereof, to a subject in need thereof.

7. A method of treating a disease, disorder or condition treatable by activating the integrated stress response via upregulation of eukaryotic initiation factor 2α (eIF2α), the method comprising administering a therapeutically effective amount of one or more compounds of claim 3 or a pharmaceutically acceptable salt, solvate and/or prodrug thereof, to a subject in need thereof.

8. The method of claim 6, wherein the disease, disorder or condition is a viral infection.

9. The method of claim 8, wherein the viral infection comprises an infection with DNA or RNA viruses.

10. The method of claim 9, wherein the DNA virus is HSV-1.

11. The method of claim 9, wherein the RNA virus is SARSCoV-2.

12. The method of claim 6, wherein the one or more compounds of claim 1 are administered by ocular, topical, transdermal, or oral administration, or by inhalation or injection.

13. The method of claim 12, wherein the one or more compounds of claim 1 are administered in combination with one or more therapeutic agents.

14. The method of claim 13, wherein the one or more therapeutic agents is an antiviral agent.

15. The method of claim 14, wherein the antiviral agent is acyclovir.

16. A method of activating the integrated stress response via upregulation of eukaryotic initiation factor 2α (eIF2α) in a cell, either in a biological sample or in a subject, comprising administering a therapeutically effective amount of one or more compounds of claim 1, or a pharmaceutically acceptable salt, solvate and/or prodrug thereof, to a cell in need thereof.

17. A method of activating the integrated stress response via upregulation of eukaryotic initiation factor 2α (eIF2α) in a cell, either in a biological sample or in a subject, comprising administering a therapeutically effective amount of one or more compounds of claim 7, or a pharmaceutically acceptable salt, solvate and/or prodrug thereof, to a cell in need thereof.


SEQUENCE LISTING (4 sequences)

SEQ ID NO: 1 - DNA, 20 bp, synthetic construct
  source 1..20: mol_type = other DNA
tcaaacctcatgggttctcc

SEQ ID NO: 2 - DNA, 20 bp, synthetic construct
  source 1..20: mol_type = other DNA
gtgtcatccaacgtggtcag

SEQ ID NO: 3 - DNA, 19 bp, synthetic construct
  source 1..19: mol_type = other DNA
acccactcctccacctttg

SEQ ID NO: 4 - DNA, 18 bp, synthetic construct
  source 1..18: mol_type = other DNA
ctcttgtgctcttgctgg